(19) United States
(12) Patent Application Publication
(10) Pub. No.: US 2008/0194571 A1
(43) Pub. Date: Aug. 14, 2008

(54) Acetylenic Piperazines as Metabotropic Glutamate Receptor Antagonists

(75) Inventors: Louise Edwards, Mississauga (CA); Mithvin Isaac, Brampton (CA); Abdelmalik Slassi, Mississauga (CA); Guang-Ri Sun, Markham (CA); Peter Dove, Toronto (CA); Erwan Arzel, Sodertalje (SE)

Correspondence Address: BIRCH, STEWART, KOLASCH & BIRCH, LLP, P.O. BOX 747, FALLS CHURCH, VA, 22040-0747, US

(73) Assignee: AstraZeneca AB, Sodertalje (SE)

(21) Appl. No.: 11/997,523

(22) PCT Filed: Aug. 4, 2006
(86) PCT No.: PCT/US2006/030394

§ 371 Date: Apr. 29, 2008

Related U.S. Application Data
(60) Provisional application No. 60/707,944, filed on Aug. 15, 2005.

(51) Int. Cl. A61K 31/4985; C07D 487/04; A61P 25/00

(52) U.S. Cl. 514/249; 544/349

(57) ABSTRACT

The invention relates to compounds of formula I or pharmaceutically acceptable salts or solvates thereof:

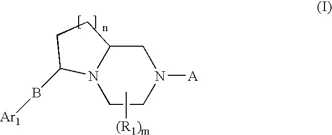

where Ar₁, A, B, R₁, m and n are as defined in the description. The invention also includes pharmaceutical compositions and uses of, and processes of making the compounds, as well as methods of medical treatment of mGluR 5 mediated disorders.

### FIELD OF THE INVENTION

The present invention relates to a new class of compounds, to pharmaceutical formulations containing said compounds and to the use of said compounds in therapy. The present invention further relates to the process for the preparation of said compounds and to new intermediates prepared therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, walking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K, Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Bailliére's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype, most particularly the mGluR5

The object of the present invention is to provide compounds exhibiting an activity at metabotropic glutamate receptors (mGluRs), especially at the mGluR5 receptor.

### SUMMARY OF THE INVENTION

One embodiment of the invention relates to compounds of formula I:

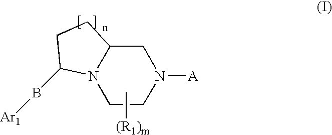

wherein:
Ar₁ is an optionally substituted aryl or heteroaryl group, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R², SO₂R², aryl, heteroaryl, cycloalkyl and heterocycloalkyl, wherein any cyclic group may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R² and SO₂R²;
A is selected from the group consisting of Ar₁, CO₂R², CONR²R³, S(O)R² and SO₂R²;
B is selected from the group consisting of vinylene and ethynylene, wherein the vinylene group is optionally substituted with up to 2 independently-selected C₁₋₆-alkyl groups;
R₁, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, (CO)R², O(CO)R², O(CO)OR², CO₂R², CONR²R³, C₁₋₆-alkyleneOR², OC₂₋₆-alkyleneOR² and C₁₋₆-alkylenecyano;
R² and R³ are independently selected from the group consisting of H, C₁₋₆-alkyl, C₁₋₆-alkylhalo, C₂₋₆-alkenyl, C₂₋₆-alkynyl and cycloalkyl;
m is an integer selected from the group consisting of 0, 1, 2, 3 and 4; and
n is an integer selected from the group consisting of 1, 2 and 3;
or a pharmaceutically-acceptable salt, hydrate, solvate, isoform, tautomer, optical isomer, or combination thereof.

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR 5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGlurR5 receptors, comprising treating a cell containing said receptor with an effective amount of the compound according to formula I.

### DETAILED DESCRIPTION OF THE PREFERRED EMBODIMENTS

The present invention is based upon the discovery of compounds that exhibit activity as pharmaceuticals, in particular as antagonists of metabotropic glutamate receptors. More particularly, the compounds of the present invention exhibit activity as antagonists of the mGluR5 receptor and, therefore, are useful in therapy, in particular for the treatment of neurological, psychiatric, pain, and gastrointestinal disorders associated with glutamate dysfunction.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “alkyl” as used herein means a straight- or branched-chain hydrocarbon radical having from one to six carbon atoms, and includes methyl, ethyl, propyl, isopropyl, t-butyl and the like.

The term “alkenyl” as used herein means a straight- or branched-chain alkenyl radical having from two to six carbon atoms, and includes ethenyl, 1-propenyl, 1-butenyl and the like.

The term “alkynyl” as used herein means a straight- or branched-chain alkynyl radical having from two to six carbon atoms, and includes 1-propynyl (propargyl), 1-butynyl and the like.

The term “cycloalkyl” as used herein means a cyclic group (which may be unsaturated) having from three to seven carbon atoms, and includes cyclopropyl, cyclohexyl, cyclohexenyl and the like.

The term “heterocycloalkyl” as used herein means a three- to seven-membered cyclic group (which may be unsaturated) having at least one heteroatom selected from the group consisting of N, S and O, and includes piperidinyl, piperazinyl, pyrrolidinyl, tetrahydrofuranyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “alkylene” as used herein means a difunctional branched or unbranched saturated hydrocarbon radical having one to six carbon atoms, and includes methylene, ethylene, n-propylene, n-butylene and the like.

The term “alkenylene” as used herein means a difunctional branched or unbranched hydrocarbon radical having two to six carbon atoms and having at least one double bond, and includes ethenylene, n-propenylene, n-butenylene and the like.

The term “alkynylene” as used herein means a difunctional branched or unbranched hydrocarbon radical having two to six carbon atoms and having at least one triple bond, and includes ethynylene, n-propynylene, n-butynylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups and includes pyridyl, indolyl, furyl, benzofuryl, thienyl, benzothienyl, quinolyl, oxazolyl and the like.

The term “cycloalkenyl” as used herein means an unsaturated cylcloaklyl group having from four to seven carbon atoms, and includes cyclopent-1-enyl, cyclohex-1-enyl and the like.

The terms “alkylaryl”, “alkylheteroaryl” and “alkylcycloalkyl” refer to an alkyl radical substituted with an aryl, heteroaryl or cycloalkyl group, and includes 2-phenethyl, 3-cyclohexyl propyl and the like.

The term “5-membered heterocyclic ring containing two or three heteroatoms independently selected from the group consisting of N, O and S” includes aromatic and heteroaromatic rings, as well as rings which may be saturated or unsaturated, and includes isoxazolyl, oxazolyl, oxadiazolyl, pyrazolyl, thiazolyl, imidazolyl, triazolyl and the like.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

### Compounds

Compounds of the invention conform generally to formula I:

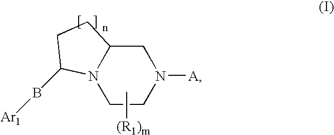

wherein Ar₁, A, B, R₁, m and n are defined hereinabove.

In one embodiment, B is an ethynylene group.

In one embodiment, Ar₁ is an optionally-substituted phenyl group; illustrative substituents may be selected from the group consisting of F, Cl, Br, nitro, C₁₋₆-alkyl, C₁₋₆-alkylhalo, OC₁₋₆-alkyl, OC₁₋₆-alkylhalo, and CN.

In another embodiment, Ar₂ is an optionally-substituted pyridyl group, for example a 2-pyridyl group; illustrative substituents may be selected from the group consisting of F, Cl, Br, nitro, C₁₋₆-alkyl, C₁₋₆-alkylhalo, OC₁₋₆-alkyl, OC₁₋₆-alkylhalo, and CN.

In still another embodiment, R¹ can be selected from the group consisting of C₁₋₆-alkyl, C₁₋₆-haloalkyl, CN, CO₂R², CONR²R³, and C₁₋₆alkyleneOR².

In one embodiment, n is 1; in another n is 2.

In yet another embodiment, m is 0; in others m is 1 or 2.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of formula I. It will further be understood that the present invention encompasses tautomers of the compounds of formula I.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example | Compound | Name |
|---|---|---|
| 12.1 | 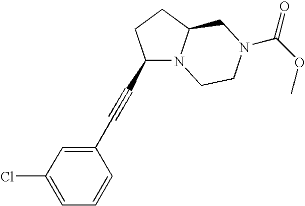 | (±)-Methyl (6R,8aS)-6-[(3-chlorophenyl)ethynyl]hexahydropyrrolo[1,2-a]pyrazine-2(1H)-carboxylate |
| 12.2 | 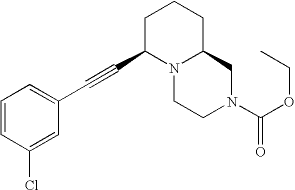 | (±)-ethyl (6R,9aS)-6-[(3-chlorophenyl)ethynyl]octahydro-2H-pyrido[1,2-a]pyrazine-2-carboxylate |
| 12.3 | 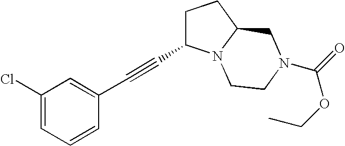 | ethyl (6S,8aS)-6-[(3-chloro-phenyl)ethynyl]hexahydropyrrolo[1,2-a]pyrazine-2(1H)-carboxylate |
| 12.4 | 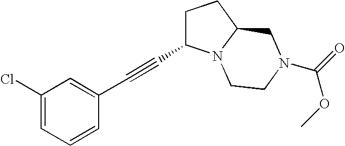 | methyl (6S,8aS)-6-[(3-chloro-phenyl)ethynyl]hexahydropyrrolo[1,2-a]pyrazine-2(1H)-carboxylate |
| 12.5 | 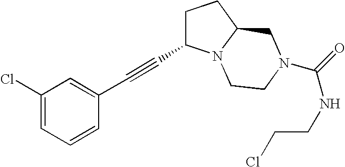 | (6S,8aS)-N-(2-chloroethyl)-6-[(3-chlorophenyl)ethynyl]hexahydropyrrolo[1,2-a]pyrazine-2(1H)-carboxamide |
| 12.6 | 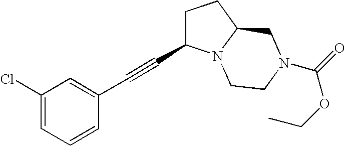 | (±)-ethyl (6R,8aS)-6-[(3-chloro-phenyl)ethynyl]hexahydropyrrolo[1,2-a]pyrazine-2(1H)-carboxylate |
| 12.7 | 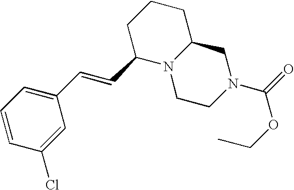 | (±)-ethyl (6R,9aS)-6-[(E)-2-(3-chlorophenyl)vinyl]octahydro-2H-pyrido[1,2-a]pyrazine-2-carboxylate |
| 13.1 | 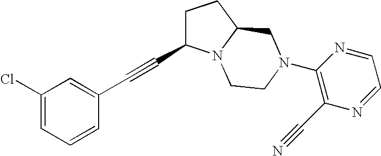 | (±)-3-[(6R,8aS)-6-[(3-chlorophenyl)ethynyl]hexahydropyrrolo[1,2-a]pyrazin-2(1H)-yl]pyrazine-2-carbonitrile |
| 13.2 | 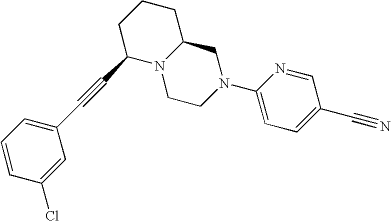 | (±)-6-{(6R,9aS)-6-[(3-chlorophenyl)ethynyl]octahydro-2H-pyrido[1,2-a]pyrazin-2-yl}nicotinonitrile |
| 13.3 | 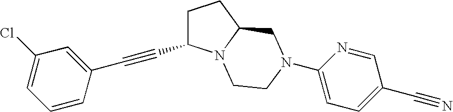 | 6-[(6S,8aS)-6-[(3-chlorophenyl)ethynyl]hexahydropyrrolo[1,2-a]pyrazin-2(1H)-yl]nicotinonitrile |
| 13.4 | 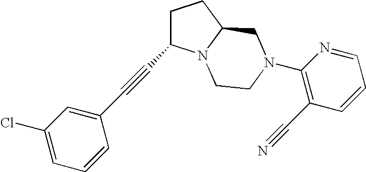 | 2-[(6S,8aS)-6-[(3-chlorophenyl)ethynyl]hexahydropyrrolo[1,2-a]pyrazin-2(1H)-yl]nicotinonitrile |
| 13.5 | 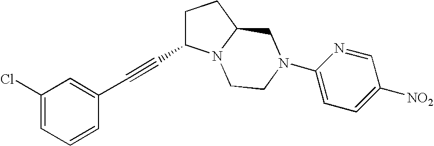 | (6S,8aS)-6-[(3-chlorophenyl)ethynyl]-2-(5-nitropyridin-2-yl)octahydropyrrolo[1,2-a]pyrazine |
| 13.6 | 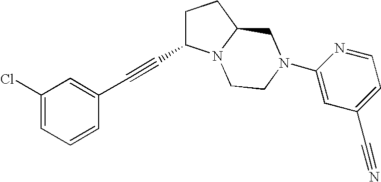 | 2-[(6S,8aS)-6-[(3-chlorophenyl)ethynyl]hexahydropyrrolo[1,2-a]pyrazin-2(1H)-yl]isonicotinonitrile |
| 13.7 | 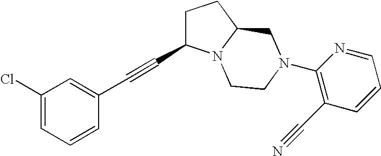 | (±)-2-[(6R,8aS)-6-[(3-chlorophenyl)ethynyl]hexahydropyrrolo[1,2-a]pyrazin-2(1H)-yl]nicotinotrile |
| 13.8 | 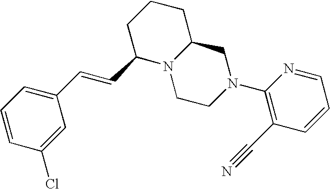 | (±)-2-{(6R,9aS)-6-[(E)-2-(3-chloro-phenyl)vinyl]octahydro-2H-pyrido[1,2-a]pyrazin-2-yl}nicotinotrile |
| 13.9 | 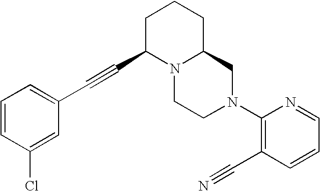 | (±)-2-{(6R,9aS)-6-[(3-chlorophenyl)ethynyl]octahydro-2H-pyrido[1,2-a]pyrazin-2-yl}nicotinotrile |
| 13.10 | 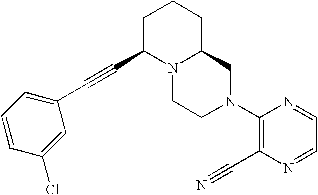 | (±)-3-{(6R,9aS)-6-[(3-chlorophenyl)ethynyl]octahydro-2H-pyrido[1,2-a]pyrazin-2-yl}pyrazine-2-carbonitrile |
| 13.11 | 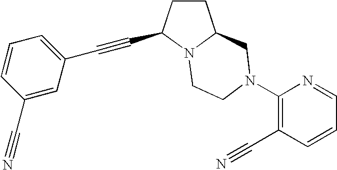 | (±)-2-[(6R,8aS)-6-[(3-cyanophenyl)ethynyl]hexahydropyrrolo[1,2-a]pyrazin-2(1H)-yl]nicotinonitrile |
| 13.12 | 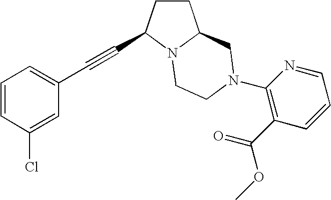 | (±)-methyl 2-[(6R,8aS)-6-[(3-chloro-phenyl)ethynyl]hexahydropyrrolo[1,2-a]pyrazin-2(1H)-yl]nicotinate |
| 13.13 | 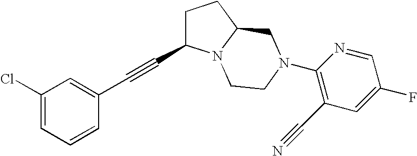 | (±)-2-[(6R,8aS)-6-[(3-chlorophenyl)ethynyl]hexahydropyrrolo[1,2-a]pyrazin-2(1H)-yl]-5-fluoro-nicotinonitrile |
| 13.14 | 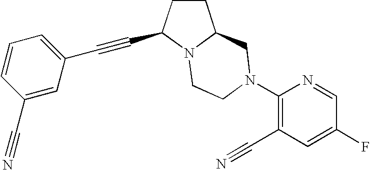 | (±)-2-[(6R,8aS)-6-[(3-cyanophenyl)ethynyl]hexahydropyrrolo[1,2-a]pyrazin-2(1H)-yl]-5-fluoro-nicotinonitrile |
| 14.1 | 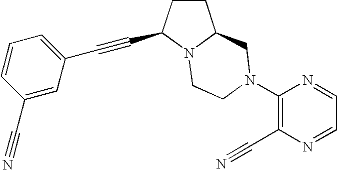 | (±)-3-[(6R,8aS)-6-[(3-cyanophenyl)ethynyl]hexahdyropyrrolo[1,2-a]pyrazin-2(1H)-yl]pyrazine-2-carbonitrile |
| 14.2 | 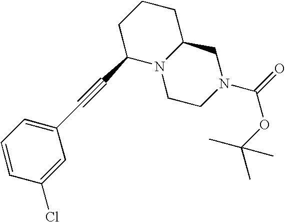 | (±)-tert-butyl (6R,9aS)-6-[(3-chlorophenyl)ethynyl]octahydro-2H-pyrido[1,2-a]pyrazine-2-carboxylate |
| 14.3 | 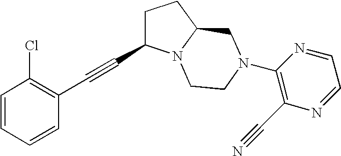 | (±)-3-[(6R,8aS)-6-[(2-chlorophenyl)ethynyl]hexahydropyrrolo[1,2-a]pyrazin-2(1H)-yl]pyrazine-2-carbonitrile |
| 14.4 | 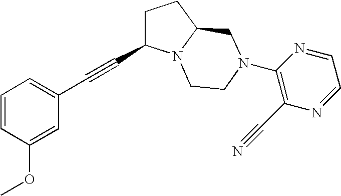 | (±)-3-[(6R,8aS)-6-[(3-methoxy-phenyl)ethynyl]hexahydropyrrolo[1,2-a]pyrazin-2(1H)-yl]-pyrazine-2-carbonitrile |
| 14.5 | 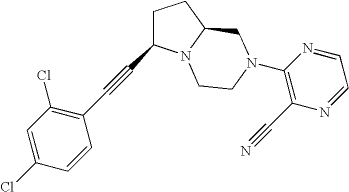 | (±)-3-[(6R,8aS)-6-[(2,4-dichloro-phenyl)ethynyl]hexahydropyrrolo[1,2-a]pyrazin-2(1H)-yl]pyrazine-2-carbonitrile |
| 14.6 | 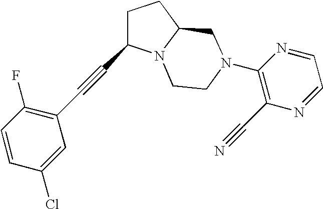 | (±)-3-[(6R,8aS)-6-[(5-chloro-2-fluorophenyl)ethynyl]hexadropyrrolo[1,2-a]pyrazin-2(1H)-yl]pyrazine-2-carbonitrile |
| 14.7 | 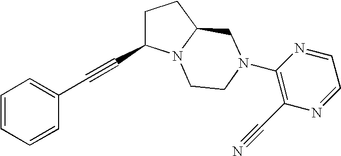 | (±)-3-[(6R,8aS)-6-(phenylethynyl)hexahydropyrrolo[1,2-a]pyrazin-2(1H)-yl]pyrazine-2-carbonitrile |
| 14.8 | 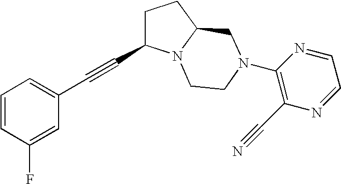 | (±)-3-[(6R,8aS)-6-[(3-fluorophenyl)ethynyl]hexahydropyrrolo[1,2-a]pyrazin-2(1H)-yl]pyrazine-2-carbonitrile |
| 14.9 | 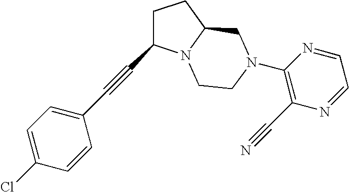 | (±)-3-[(6R,8aS)-6-[(4-chlorophenyl)ethynyl]hexahydropyrrolo[1,2-a]pyrazin-2(1H)-yl]pyrazine-2-carbonitrile |
| 14.10 | 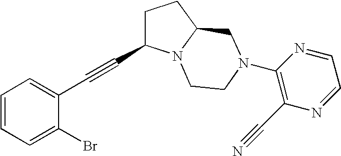 | (±)-3-[(6R,8aS)-6-[(2-bromophenyl)ethynyl]hexahydropyrrolo[1,2-a]pyrazin-2(1H)-yl]pyrazine-2-carbonitrile |
| 14.11 | 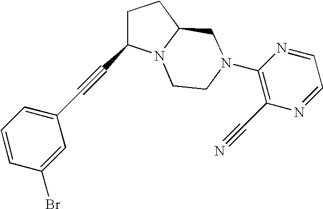 | (±)-3-[(6R,8aS)-6-[(3-bromophenyl)ethynyl]hexahydropyrrolo[1,2-a]pyrazin-2(1H)-yl]pyrazine-2-carbonitrile |
| 14.12 | 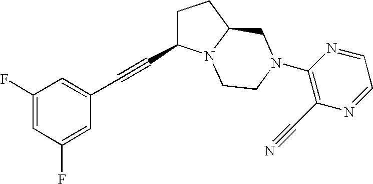 | (±)-3-[(6R,8aS)-6-[(3,5-difluoro-phenyl)ethynyl]hexahydropyrrolo[1,2-a]pyrazin-2(1H)-yl]pyrazine-2-carbonitrile |
| 14.13 | 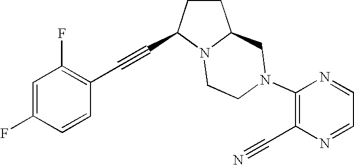 | (±)-3-[(6R,8aS)-6-[(2,4-difluoro-phenyl)ethynyl]hexahydropyrrolo[1,2-a]pyrazin-2(1H)-yl]pyrazine-2-carbonitrile |
| 14.14 | 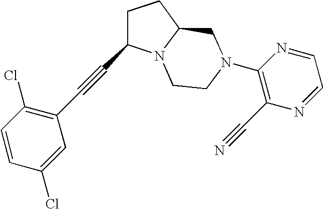 | (±)-3-[(6R,8aS)-6-[(2,5-dichloro-phenyl)ethynyl]hexahydropyrrolo[1,2-a]pyrazin-2(1H)-yl]pyrazine-2-carbonitrile |
| 14.15 | 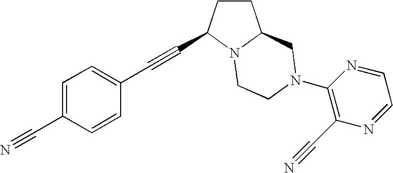 | (±)-3-[(6R,8aS)-6-[(4-cyanophenyl)ethynyl]hexahydropyrrolo[1,2-a]pyrazin-2(1H)-yl]pyrazine-2-carbonitrile |
| 14.16 | 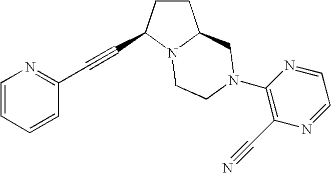 | (±)-3-[(6R,8aS)-6-[(pyridin-2-yl-ethynyl]hexahydropyrrolo[1,2-a]pyrazin-2(1H)-yl]pyrazine-2-carbonitrile |
| 14.17 | 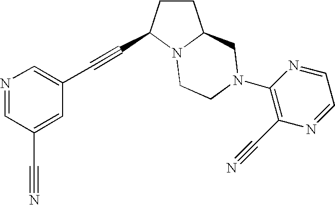 | (±)-3-[(6R,8aS)-6-[(5-cyanopyridin-3-yl)ethynyl]hexahydropyrrolo[1,2-a]pyrazin-2(1H)-yl]pyrazine-2-carbonitrile |
| 14.18 | 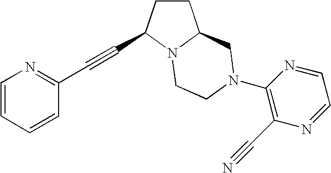 | (±)-2-[(6R,8aS)-6-(pyridin-2-yl-ethynyl)hexahydropyrrolo[1,2-a]pyrazin-2(1H)-yl]nicotinonitrile |
| 14.19 | 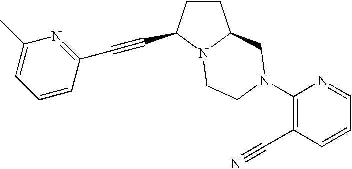 | (±)-2-[(6R,8aS)-6-[(6-methylpryridin-2-yl)ethynyl]hexahydropyrrolo[1,2-a]pyrazin-2(1H)-yl]nicotinonitrile |
| 14.20 | 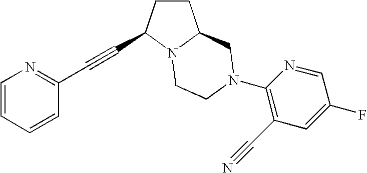 | (±)-5-fluoro-2-[(6R,8aS)-6-(pyridin-2-ylethynyl)hexahydropyrrolo[1,2-a]pyrazin-2(1H)-yl]nicotinonitrile |
| 14.21 | 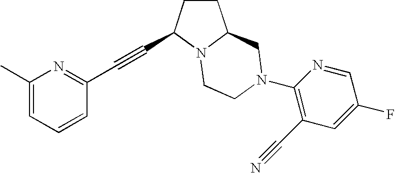 | (±)-5-fluoro-2-[(6R,8aS)-6-[(6-methylpyridin-2-yl)ethynyl)hexahydropyrrolo[1,2-a]pyrazin-2(1H)-yl]nicotinonitrile |
| 14.22 | 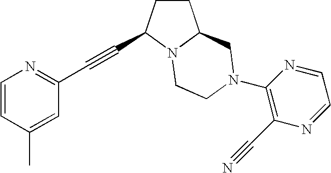 | (±)-3-[(6R,8aS)-6-[(4-methylpyridin-2-yl)ethynyl)hexahydropyrrolo[1,2-a]pyrazin-2(1H)-yl]pyrazine-2-carbonitrile |
| 14.23 | 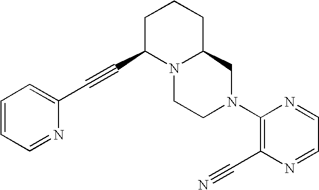 | (±)-3-[(6R,9aS)-6-(pyridin-2-yl-ethynyl)octahydro-2H-pyrido[1,2-a]pyrazine-2-carbonitrile |
| 14.24 | 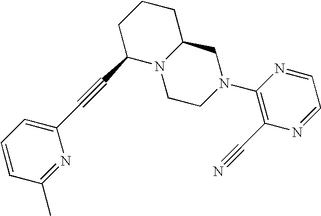 | (±)-3-{(6R,9aS)-6-[(6-methylpyridin-2-yl)ethynyl]octahydro-2H-pyrido[1,2-a]pyrazin-2-yl}pyrazine-2-carbonitrile |
| 14.25 | 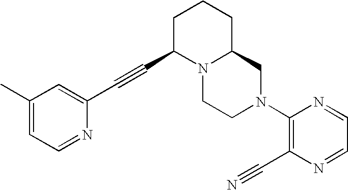 | (±)-3-{(6R,9aS)-6-[(4-methylpyridin-2-yl)ethynyl]octahydro-2H-pyrido[1,2-a]pyrazin-2-yl}pyrazine-2-carbonitrile |
| 14.26 | 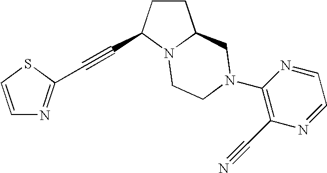 | (±)-3-[(6R,8aS)-6-(1,3-thiazol-2-yl-ethynyl)hexahydropyrrolo[1,2-a]pyrazin-2(1H)-yl]pyrazine-2-carbonitrile |
| 14.27 | 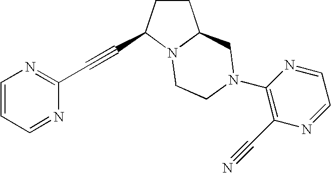 | (±)-3-[(6R,8aS)-6-(pyrimidin-2-yl-ethynyl)hexahydropyrrolo[1,2-a]pyrazin-2(1H)-yl]pyrazine-2-carbonitrile |
| 14.28 | 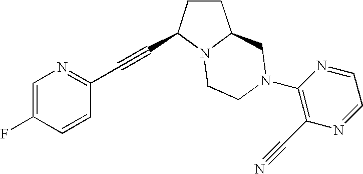 | (±)-3-[(6R,8aS)-6-[(5-fluoropyridin-2-yl)ethynyl]hexahydropyrrolo[1,2-a]pyrazin-2(1H)-yl]pyrazine-2-carbonitrile |
| 14.29 | 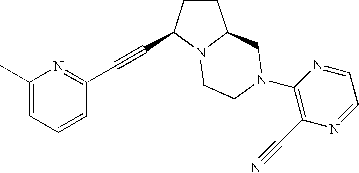 | (±)-3-[(6R,8aS)-6-[(6-methylpyridin-2-yl)ethynyl]hexahydropyrrolo[1,2-a]pyrazin-2(1H)-yl]pyrazine-2-carbonitrile |
| 14.30 | 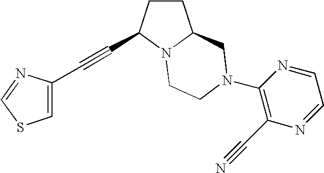 | (±)-3-[(6R,8aS)-6-(1,3-thiazol-4-ylethynyl)hexahydropyrrolo[1,2-a]pyrazin-2(1H)-yl]pyrazine-2-carbonitrile |
| 15 | 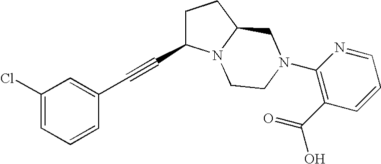 | (±)-2-[(6R,8aS)-6-[(3-chlorophenyl)ethynyl]hexahydropyrrolo[1,2-a]pyrazin-2(1H)-yl]nicotinic acid |
| 16 | 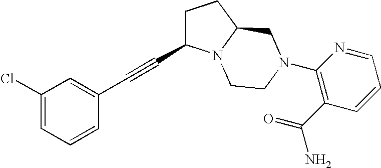 | (±)-2-[(6R,8aS)-6-[(3-chlorophenyl)ethynyl]hexahydropyrrolo[1,2-a]pyrazin-2(1H)-yl]nicotinamide |
| 17 | 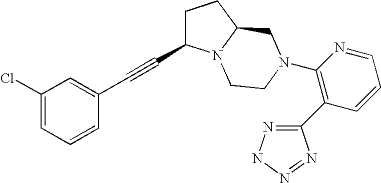 | (±)-[(6R,8aS)-6-[(3-chlorophenyl)ethynyl]-2-[3-(2H-tetrazol-5-yl)pyridin-2-yl]octahydropyrrolo[1,2-a]pyrazine |
| 18.1 | 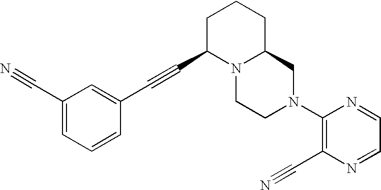 | (±)-3-{(6R,9aS)-6-[(3-chlorophenyl)ethynyl]octahydro-2H-pyrido[1,2-a]pyrazin-2-yl}pyrazine-2-carbonitrile |
| 18.2 | 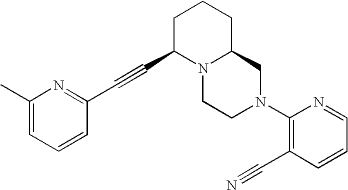 | (±)-2-{(6R,8aS)-6-[(6-methylpyridin-2-yl)ethynyl]octahydro-2H-pyrido[1,2-a]pyrazin-2-yl}nicotinonitrile |
| 18.3 | 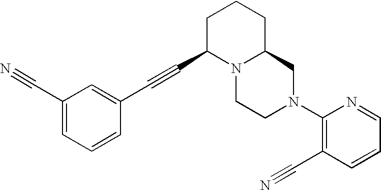 | (±)-2-{(6R,9aS)-6-[(3-cyanophenyl)ethynyl]octahydro-2H-pyrido[1,2-a]pyrazin-2-yl}nicotinonitrile |
| 18.4 | 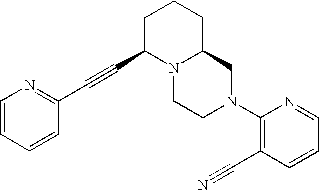 | (±)-2-{(6R,9aS)-6-(pyridin-2-yl-ethynyl)octahydro-2H-pyrido[1,2-a]pyrazin-2-yl}nicotinonitrile |
| 18.5 | 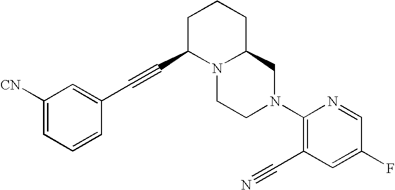 | (±)-2-{(6R,9aS)-6-[(3-cyanophenyl)ethynyl]octahydro-2H-pyrido[1,2-a]pyrazin-2-yl}-5-fluoronicotinonitrile |
| 18.6 | 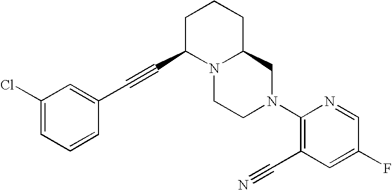 | (±)-2-{(6R,9aS)-6-[(3-chlorophenyl)ethynyl]octahydro-2H-pyrido[1,2-a]pyrazin-2-yl}nicotinonitrile |
| 18.7 | 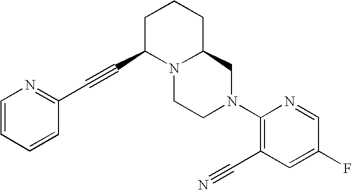 | (±)-5-fluoro-2-[(6R,9aS)-6-(pyridin-2-ylethynyl)octahydro-2H-pyrido[1,2-a]pyrazin-2-yl]nicotinonitrile |
| 18.8 | 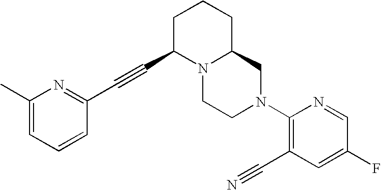 | (±)-5-fluoro-2-{(6R,9aS)-6-[(6-methylpyridin-2-yl)ethynyl]octahydro-2H-pyrido[1,2-a]pyrazin-2-yl}nicotinonitrile |
| 20.1 | 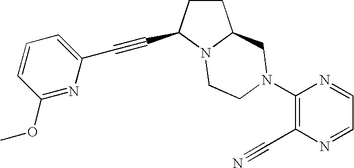 | (±)-3-[(6R,8aS)-6-[(6-methoxypyridin-2-yl)ethynyl]hexahydropyrrolo[1,2-a]pyrazin-2(1H)-yl]pyrazine-2-carbonitrile |
| 20.2 | 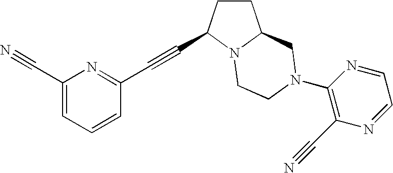 | (±)-3-[(6R,8aS)-6-[(6-cyanopyridin-2-yl)ethynyl]hexahydropyrrolo[1,2-a]pyrazin-2(1H)-yl]pyrazine-2-carbonitrile |
| 20.3 | 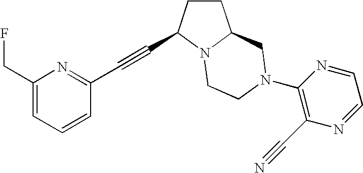 | (±)-3-[(6R,8aS)-6-{[(6-(fluoromethyl)pyridin-2-yl]ethynyl}hexahydropyrrolo[1,2-a]pyrazin-2(1H)-yl]pyrazine-2-carbonitrile |
| 21.1 | 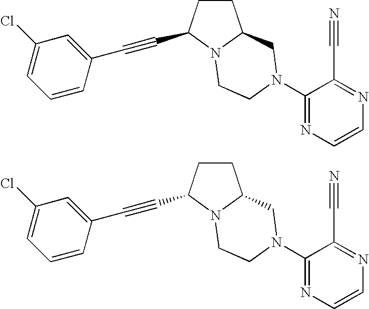 | 3-[(6R,8aS)-6-[(3-chlorophenyl)ethynyl]hexahydropyrrolo[1,2-a]pyrazin-2(1H)-yl]pyrazine-2-carbonitrile&3-[(6S,8aR)-6-[(3-chlorophenyl)ethynyl]hexahydropyrrolo[1,2-a]pyrazin-2(1H)-yl]pyrazine-2-carbonitrile |
| 21.2 | 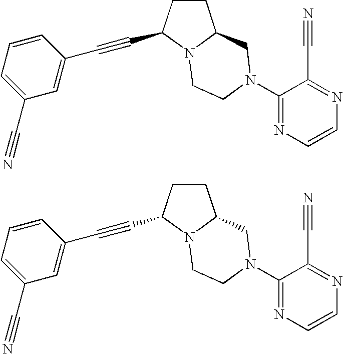 | 3-[(6R,8aS)-6-[(3-cyanophenyl)ethynyl]hexahydropyrrolo[1,2-a]pyrazin-2(1H)-yl]pyrazine-2-carbonitrile&3-[(6S,8aR)-6-[(3-cyanophenyl)ethynyl]hexahydropyrrolo[1,2-a]pyrazin-2(1H)-yl]pyrazine-2-carbonitrile |
| 21.3 | 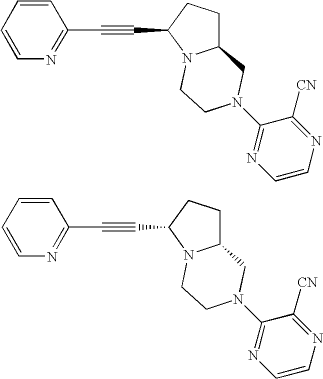 | 3-[(6R,8aS)-6-(pyridin-2-ylethynyl)hexahydro-pyrrolo[1,2-a]pyrazin-2(1H)-yl]pyrazine-2-carbonitrile&3-[(6S,8aR)-6-(pyridin-2-ylethynyl)hexahydro-pyrrolo[1,2-a]pyrazin-2(1H)-yl]pyrazine-2-carbonitrile |
| 21.4 | 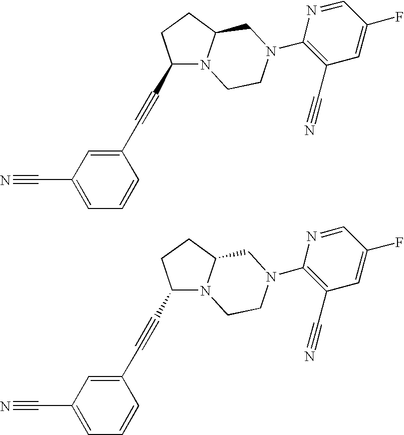 | 2-[(6R,8aS)-6-[(3-cyanophenyl)ethynyl]hexahydropyrrolo[1,2-a]pyrazin-2(1H)-yl]-5-fluoronicotinonitrile&2-[(6S,8aR)-6-[(3-cyanophenyl)ethynyl]hexahydropyrrolo[1,2-a]pyrazin-2(1H)-yl]-5-fluoronicotinonitrile |
| 21.5 | 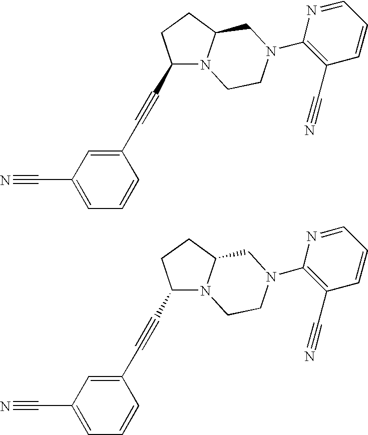 | 2-[(6R,8aS)-6-[(3-cyanophenyl)ethynyl]hexahydropyrrolo[1,2-a]pyrazin-2(1H)-yl]nicotinonitrile&2-[(6S,8aR)-6-[(3-cyanophenyl)ethynyl]hexahydropyrrolo[1,2-a]pyrazin-2(1H)-yl]nicotinonitrile |
| 21.6 | 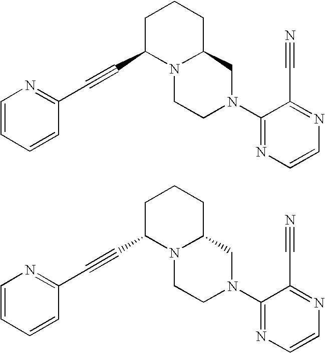 | 3-[(6R,9aS)-6-pyridin-2-ylethynyl)octahydro-2H-pyrido[1,2-a]pyrazin-2-yl]pyrazine-2-carbonitrile&3-[(6S,9aR)-6-pyridin-2-ylethynyl)octahydro-2H-pyrido[1,2-a]pyrazin-2-yl]pyrazine-2-carbonitrile |
| 21.7 | 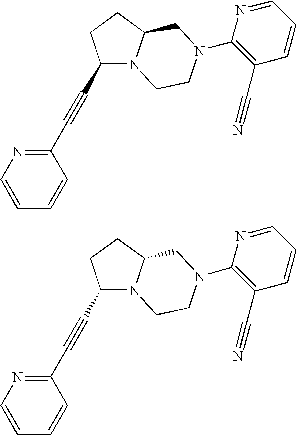 | 2-[(6R,8aS)-6-(pyridin-2-ylethynyl)hexahydropyrrolo[1,2-a]pyrazin-2(1H)-yl]nicotinonitrile&2-[(6S,8aR)-6-(pyridin-2-ylethynyl)hexahydropyrrolo[1,2-a]pyrazin-2(1H)-yl]nicotinonitrile |
| 21.8 | 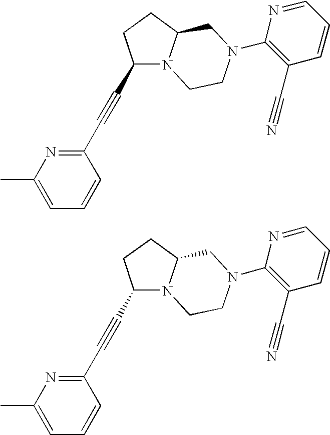 | 2-[(6R,8aS)-6-[(6-methylpyridin-2-yl)ethynyl]hexahydropyrrolo[1,2-a]pyrazin-2(1H)-yl]nicotinonitrile&2-[(6S,8aR)-6-[(6-methylpyridin-2-yl)ethynyl]hexahydropyrrolo[1,2-a]pyrazin-2(1H)-yl]nicotinonitrile |
| 21.9 | 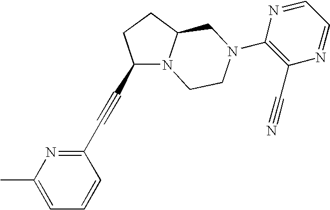 | 3-[(6R,8aS)-6-[(6-methylpyridin-2-yl)ethynyl]hexahydropyrrolo[1,2-a]pyrazin-2(1H)-yl]pyrazine-2-carbonitrile&3-[(6S,8aR)-6-[(6-methylpyridin-2-yl)ethynyl]hexahydropyrrolo[1,2-a]pyrazin-2(1H)-yl]pyrazine-2-carbonitrile |
| | 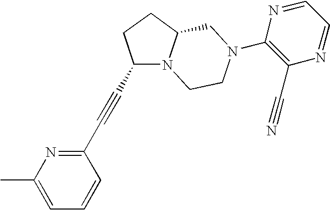 | |
| 21.10 | 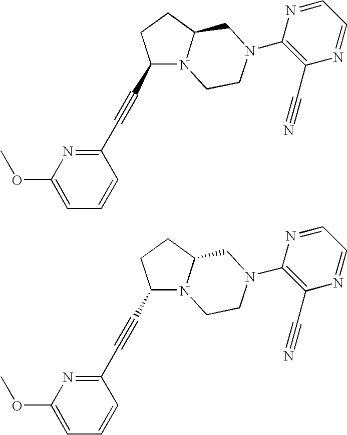 | 3-[(6R,8aS)-6-[(6-methoxypyridin-2-yl)ethynyl]hexahydropyrrolo[1,2-a]pyrazin-2(1H)-yl]pyrazine-2-carbonitrile&3-[(6S,8aR)-6-[(6-methoxypyridin-2-yl)ethynyl]hexahydropyrrolo[1,2-a]pyrazin-2(1H)-yl]pyrazine-2-carbonitrile |
| 21.11a | 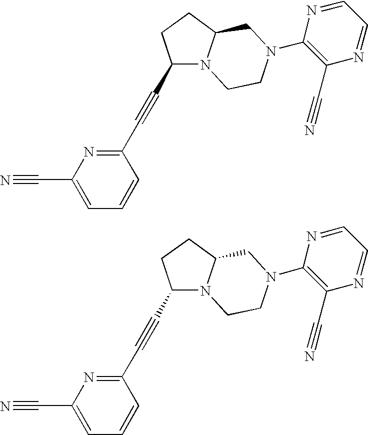 | 3-[(6R,8aS)-6-[(6-cyanopyridin-2-yl)ethynyl]hexahydropyrrolo[1,2-a]pyrazin-2(1H)-yl]pyrazine-2-carbonitrile&3-[(6S,8aR)-6-[(6-cyanopyridin-2-yl)ethynyl]hexahydropyrrolo[1,2-a]pyrazin-2(1H)-yl]pyrazine-2-carbonitrile |
| 21.12 | 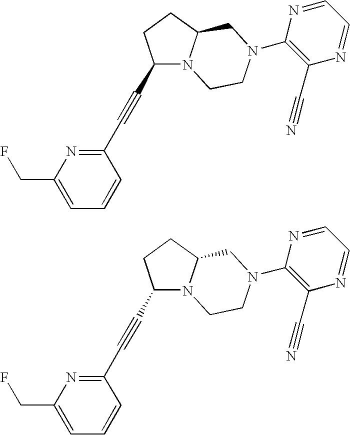 | 3-[(6R,8aS)-6-{[(6-fluoromethyl)pyridin-2-yl]ethynyl}hexahydropyrrolo[1,2-a]pyrazin-2(1H)-yl]pyrazine-2-carbonitrile&3-[(6S,8aR)-6-{[(6-fluoromethyl)pyridin-2-yl]ethynyl}hexahydropyrrolo[1,2-a]pyrazin-2(1H)-yl]pyrazine-2-carbonitrile |

### Pharmaceutical Composition

The compounds of the present invention may be formulated into conventional pharmaceutical composition comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout.

The invention relates to compounds of Formula I as defined hereinbefore, for use in treatment of stroke, head trauma, anoxic and ischemic injuries, hypoglycemia, cardiovascular diseases and epilepsy.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a Formula I compound for the manufacture of a medicament for inhibition of transient lower esophageal sphincter relaxations, for the treatment of GERD, for the prevention of G.I. reflux, for the treatment regurgitation, for treatment of asthma, for treatment of laryngitis, for treatment of lung disease, for the management of failure to thrive, for the treatment of irritable bowel disease (IBS) and for the treatment of functional dyspepsia (FD).

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

### Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

### Process of Preparation

Another aspect of the present invention provides processes for preparing compounds of Formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It also is to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, normal and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The RS/SR diastereomer of the bicyclic intermediate wherein n=1 may be prepared according to the method shown in Scheme 1, below, by treatment of the meso-dibromide a with an ethylene diamine. Reduction of the amide and ester groups may be done in one pot with a reducing agent such as LAH to give the bicyclic piperazine alcohol c. The free NH of the piperazine may displace a halide atom such as chloride from a heteroaromatic such as pyridine or pyrazine to introduce the A moiety of formula I at this stage, or the NH may be protected with a group such as BOC to allow later introduction of the A group at a later stage following deprotection.

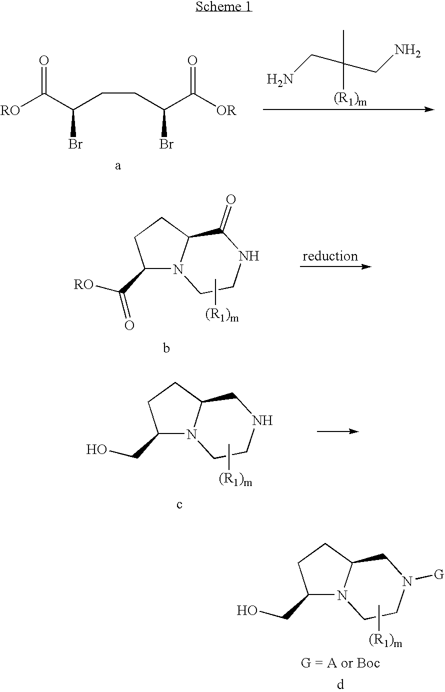

The homologated bicyclic piperazine alcohol wherein n=2 may be prepared according to the method shown in Scheme 2, beginning with reduction of pyridyl diester e to the piperidine diester f. Diketopiperazine formation may be done via acylation with a protected alpha-amino acid, deprotection and cyclization; or acylation with an alpha bromoacid halide followed by cyclization using ammonia as a source of the piperazine N atom. The ester and amide moieties in the diketopiperazine may be simultaneous reduced to provide the bicyclic piperazine alcohol h. As above, arylation to introduce A onto the free NH of the piperazine moiety may be done at this stage, or the NH may be protected and A introduced at a later stage following deprotection.

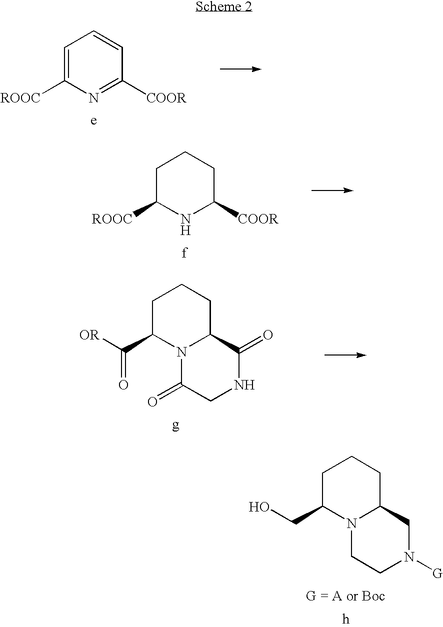

The SS enantiomeric bicyclic intermediate wherein n=1 may be prepared according to the method shown in Scheme 3, below, using a protected pyroglutamic acid derivative from the chiral pool. The lactam group may be converted to the lactol i by reduction with a reducing reagent such as lithium triethylborohydride. Treatment with an alcohol such as methanol in the presence of a mild acid such as toluenesulfonic acid may be used to convert the OH to an alkoxy leaving group, which may be used to introduce a olefin moiety by treatment with a vinyl metallic species such as vinyl magnesium bromide or propenyllithium with a copper salt such as CuBr.Me₂S and BF₃.Et₂O. Ozonolysis of the vinyl group followed by workup with a reagent such as Me₂S may be used to obtain the aldehyde, which may be reduced in a subsequent step to the bicyclic piperazine alcohol m to facilitate the subsequent introduction of the heteroaryl moiety A or a protecting group for the piperazine NH. The RR enantiomers may also be prepared in a similar manner.

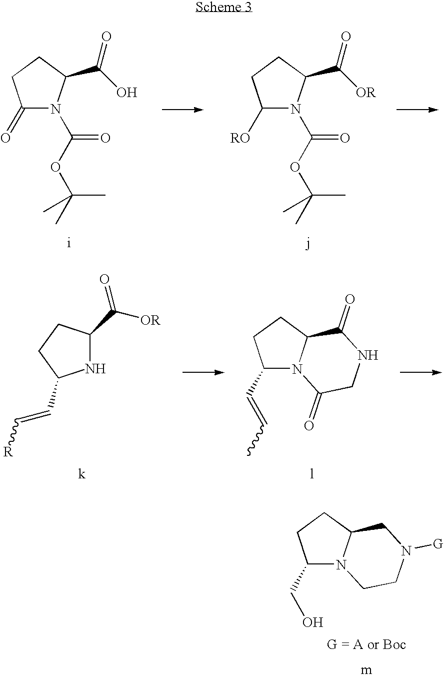

Compounds of Formula I wherein B is an acetylene may be prepared by the methods shown in Scheme 4, below. Oxidation of these bicyclic piperazine alcohols n to the corresponding aldehydes o may be accomplished under mild conditions such as Swern oxidation, followed by conversion to the corresponding terminal acetylenes p using a diazo-phosphonate under mildly basic conditions in a protic solvent such as methanol. The terminal acetylenes may be coupled to an aryl iodide or aryl bromide using palladium and copper catalysts such as Pd(PPh₃)₂Cl₂ with CuI in the presence of an amine base such as Et₃N to yield compounds q.

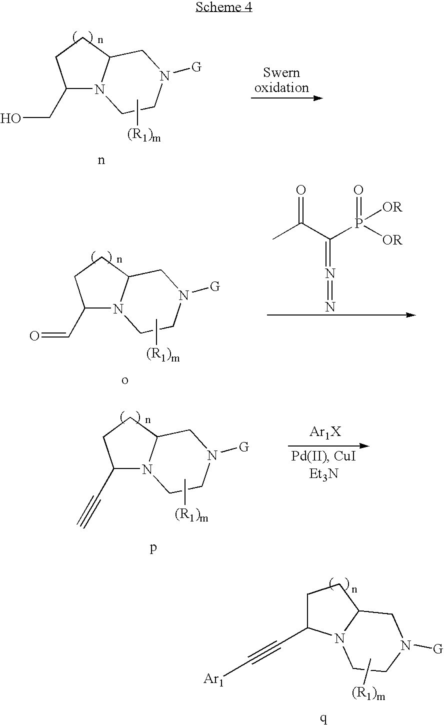

Compounds of Formula I wherein B is an E-olefin may be prepared by the methods shown in Scheme 5, below. Olefination of the bicyclic piperazine aldehydes o may be accomplished using a stabilized Witting reagent generated from a benzyl triphenylphosphonium bromide and a strong base such as nBuLi in a solvent such as THF at low temperature (−78 to −20° C.) to yield compounds r.

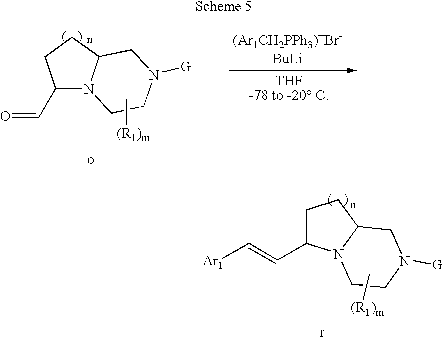

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### General Methods

All starting materials are commercially available or earlier described in the literature.

The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Unless otherwise indicated, in the tables below ¹H NMR data was obtained at 300 MHz, using CDCl₃ as the solvent.

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.).

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in an Emrys Optimizer from Biotage/Personal Chemistry or a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca²⁺]ᵢ mobilization was done 24 hours after seeding.

FLIPR experiments were done using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 min. with 10 mM LiCl. Compounds (agonists) were added and incubated for 30 min. at 37° C. Antagonist activity was determined by pre-incubating test compounds for 15 min., then incubating in the presence of glutamate (80 μM) or DHPG (30 μM) for 30 min. Reactions were terminated by the addition of perchloric acid (5%). Samples were collected and neutralized, and inositol phosphates were separated using Gravity-Fed Ion-Exchange Columns.

### General Methods

### Abbreviations

BOC tert-butoxycarbonyl

### BSA Bovine Serum Albumin

### CCD Charge Coupled Device

### CRC Concentration Response Curve

DCM dichloromethane
DBPG 3,5-dihydroxyphenylglycine;

### DMF N,N-dimethylformamide

DMSO dimethyl sulfoxide

### EDTA Ethylene Diamine Tetraacetic Acid

Et₃N triethylamine
FLIPR Fluorometric Imaging Plate reader
GHEK Human Embryonic Kidney expressing Glutamate Transporter
HEPES 4-(2-hydroxyethyl)-1-piperazineethanesulfonic acid (buffer)
IP3 inositol triphosphate
MeOH methanol
NMR nuclear magnetic resonance
ppm parts per million
RT room temperature
SPE solid phase extraction
TFA trifluoroacetic acid
THF tetrahydrofuran

### Example 1

### (±)-Ethyl (6R,8aS)-1-oxooctahydropyrrolo[1,2-a]pyrazine-6-carboxylate

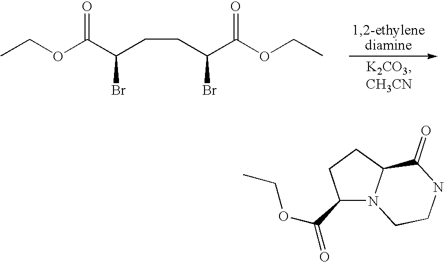

To a mixture of 1,2-ethylene diamine (20 mL, 0.28 mol), K₂CO₃ (40 g, 0.29 mol) and acetonitrile (300 mL) was added slowly a solution of diethyl meso-2,5-dibromoadipate (50 g, 0.139 mol) in acetonitrile (200 mL) over 36 h at RT. The solvent was removed and DCM (300 mL) was added. After filtration, the DCM was evaporated to afford the crude product (32 g, purity >90%). ¹H NMR (300 MHz, CDCl₃): δ (ppm) 1.30 (t, 3H), 1.96-2.18 (m, 4H), 2.52 (m, 1H), 2.94 (m, 1H), 3.15 (m, 1H), 3.35 (m, 2H), 3.60 (m, 1H), 4.23 (q, 2H), 6.12 broad, 1H).

### Example 2

### (±)-(6R,8aS)-Octahydropyrrolo[1,2-a]pyrazin-6-ylmethanol

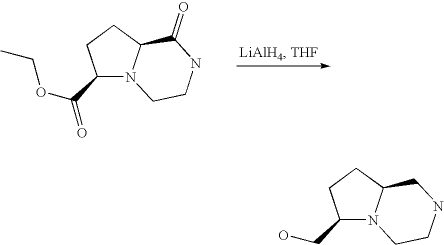

To a suspension of LiAlH₄ (16 g, 0.42 mol) in THF (350 mL) was added a solution of (±)-ethyl (6R,8aS)-1-oxooctahydropyrrolo[1,2-a]pyrazine-6-carboxylate (32 g) in THF (150 mL) at 0° C. over 30 min. The reaction mixture was stirred at RT overnight and at 80° C. for 2 h. To the resulting mixture was added carefully NaOH aq (10% 18 mL) over 30 min at 0° C. After stirring for additional 30 min, the mixture was filtered through Celite® and the filtrate was concentrated to afford the crude aminoalcohol (20.5 g, purity >85%). ¹H NMR (300 MHz, CDCl₃): δ (ppm) 1.28 (m, 1H), 1.76-1.87 (m, 3H), 2.15 (m, 2H), 2.45 (m, 2H), 2.76 (m, 1H), 3.01 (m, 2H), 3.13 (m, 1H), 3.46 (broad d, 1H), 3.70-3.79 (m, 2H).

### Example 3

### (±)-tert-Butyl (6R,8aS)-6-(hydroxymethyl)hexahydropyrrolo[1,2-a]pyrazine-2(1H)-carboxylate

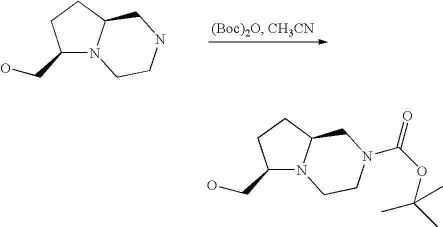

To a solution of (±)-(6R,8aS)-octahydropyrrolo[1,2-a]pyrazin-6-ylmethanol (9 g, crude) in acetonitrile (120 mL) was added (Boc)₂O (13.5 g, 62 mmol) at 0° C. over 10 min. The mixture was stirred at RT for 2 h. To the resulting mixture was added Na₂CO₃ aq (sat. 200 mL) and extracted with ethyl acetate (180 mL×3). The combined extract was dried and the solvent was removed with rotary evaporator to give residue which was purified on silica gel column to afford boc-protected alcohol (8 g, 64%). ¹H NMR (300 MHz, CDCl₃): δ (ppm) 1.28 (m, 1H), 1.48 (s, 9H), 1.79 (m, 3H), 2.06 (m, 2H), 2.53 (m, 2H), 2.65-3.00 (m, 2H), 3.58 (m, 1H), 4.13 (m, 1H), 4.12 (broad, 2H).

### Example 4

### 1-tert-butyl 2-methyl (2S,5S)-5-[(1E/Z)-prop-1-en-1-yl]pyrrolidine-1,2-dicarboxylate

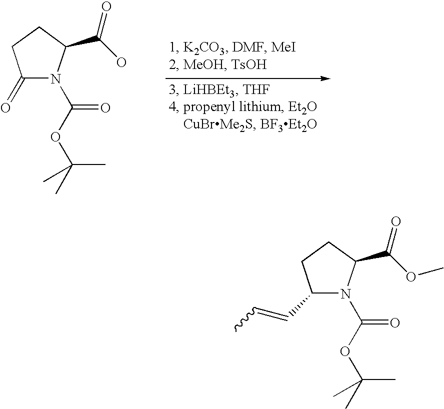

A mixture of (2S)-1-(tert-butoxycarbonyl)-5-oxopyrrolidine-2-carboxylic acid (9.7 g, 42 mmol), K₂CO₃ (6.6 g, 48 mmol) and DMF (80 mL) was stirred at RT for 20 min. MeI (5.5 mL, 88 mmol) was added and this mixture was stirred overnight, diluted with ethyl acetate (600 mL) and washed with H₂O (300 mL×3). The organic layer was dried over Anhydrous sodium sulphate and the solvent was removed to give methyl ester. The ester was diluted with dry THF (100 mL) and cooled to −78° C. LiHBEt₃ THF solution (1N, 48 mL, 48 mmol) was slowly added to above system over 15 min at −78° C. The mixture was stirred for 1 h and was poured into NaHCO₃ aq (sat, 200 mL), followed by addition of H₂O₂ (30%, 2 mL). This resulting mixture was stirred for 1 h at 0° C. and extracted with ethyl acetate (200 mL×3). The extract was dried over Anhydrous sodium sulphate and the solvent was removed. The residue was treated with dry MeOH (150 mL) and 4-methylbenzenesulfonic acid (2 g). The resulting mixture was stirred overnight at RT, NaHCO₃(aq) (sat. 50 mL) was added and the product was extracted with DCM (150 mL×3). Removal of solvent gave intermediate (9.7 g).

To a suspension of CuBrMe₂S (16.8 g, 76 mmol) and Et₂O (100 mL) was added a solution of propenyl lithium, generated from propenyl bromide (6.5 mL, 76 mmol), lithium metal (1.8 g, 260 mmol) and Et₂O (100 mL) at RT for 1 h, at −40° C. over 10 min. After stirring for 1 h at −40° C., the reaction mixture was cooled to −78° C. and BF₃.Et₂O (11.5 mL, 90 mmol) was added over 5 min. The resulting mixture was stirred at −78° C. for 1 h. To this reaction mixture was added a solution of intermediate obtained above in Et₂O (100 mL). After the mixture was allowed to warm to 0° C. over 3.5 h, it was poured into saturated NH₄Cl_{(aq)}/NH₄OH (1:1) solution (200 mL) and stirred for 30 min. The organic phase was separated and water phase was extracted with Et₂O (150 mL×2). The combined extracts were dried and the solvent was removed to provide product (10 g, purity >88%). ¹H NMR (300 MHz, CD₃OD): δ (ppm) 1.43 and 1.50 (s, 9H), 1.63-2.28 (m, 7H), 3.73 (m, 3H), 4.30-4.88 (m, 2H), 5.37-5.58 (m, 2H).

### Example 5

### (6S,8aS)-6-[(1E/Z)-prop-1-en-1-yl]hexahydropyrrolo[1,2-a]pyrazine-1,4-dione

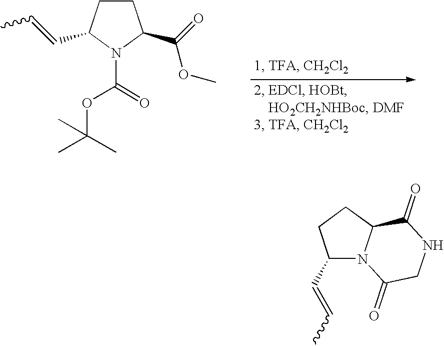

To a mixture of 1-tert-butyl 2-methyl (2S,5S)-5-[(1E/Z)-prop-1-en-1-yl]pyrrolidine-1,2-dicarboxylate (10 g, 37 mmol) and DCM (75 mL) was added TFA (25 mL) at 0° C. The mixture was stirred at RT for 2 h. After removal of DCM and TFA, the residue was diluted with ethyl acetate and washed with Na₂CO₃ aq. Organic solution was dried over Anhydrous sodium sulphate and the solvent was removed to afford amine. The intermediate amine was treated with EDCI (7.8 g, 40 mmol), HOBT (5.8 g, 43 mmol), HOCOCH₂N(boc) (7.3 g, 42 mmol) in DMF (100 mL) at RT overnight. The product was extracted into ethyl acetate and washed with NaCl aq (sat.) and water successively. The organic phase was concentrated to give amide, which was treated with TFA (25 mL) in DCM (75 mL) for 1 h at RT. DCM and TFA were removed and the residue was treated with Na₂CO₃ aq (sat.) to adjust pH value about 8 and extracted with DCM (3×300 mL). The extracts were dried and the solvent was removed to give solid, which was triturated with Et₂O and hexane to produce pure product (4 g, 47%). ¹H NMR (300 MHz, CD₃OD): δ (ppm) 1.47-1.83 (m, 4H), 2.05-2.28 (m, 3H), 3.82-3.89 (m, 1H), 4.07-4.21 (m, 2H), 4.65-4.95 (m, 1H), 5.31-5.66 (m, 2H), 6.86 (broad, 1H);

### Example 6

### Tert-butyl (6S,8aS)-6-(hydroxymethyl)hexahydropyrrolo[1,2-a]pyrazine-2(1H)-carboxylate

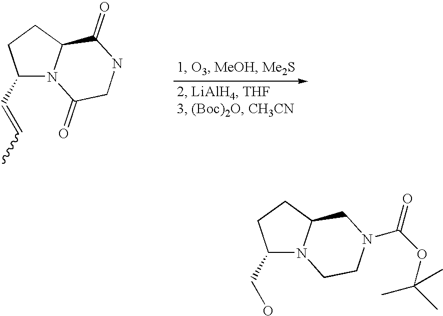

To a solution of (6S,8aS)-6-[(1E/Z)-prop-1-en-1-yl]hexahydropyrrolo[1,2-a]pyrazine-1,4-dione (2.8 g, 14.4 mmol) in MeOH (100 mL) was bubbled O₃ at −78° C. for 20 min. Me₂S (4 mL) was added and the resulting mixture was stirred at RT overnight. The solvent was removed and the residue was treated with LiAlH₄ (2.6 g, 70 mmol) and THF (160 mL) at RT overnight and at 80° C. for 2 h. To the resulting mixture was added carefully NaOH_{(aq)} (10% 5 mL) over 30 min at 0° C. After stirring for additional 30 min, the mixture was filtered through Celite® and the filtrate was concentrated to afford the crude amino-alcohol (1.8 g, crude). Et₃N (1 mL) and (Boc)₂O (2.66 g, 12 mmol) were added to the crude amino-alcohol in DCM (15 mL) at 0° C., and the resulting mixture was stirred for 2 h. After washing with saturated Na₂CO_{3(aq)}, the organic solution was dried, concentrated and purified on silica gel column to afford boc-protected amino-alcohol (638 mg, 18%). ¹H NMR (300 MHz, CDCl₃): δ (ppm) 1.48 (s, 9H), 1.65-2.11 (m, 4H), 2.55 (broad, 1H), 2.76-3.24 (m, 6H), 3.44-3.70 (m, 4H).

### Example 7

### (±)-Tert-butyl (6R,9aS)-6-(hydroxymethyl)octahydro-2H-pyrido[1,2-a]pyrazine-2-carboxylate

### i) Dimethyl (2R,6S)-piperidine-2,6-dicarboxylate

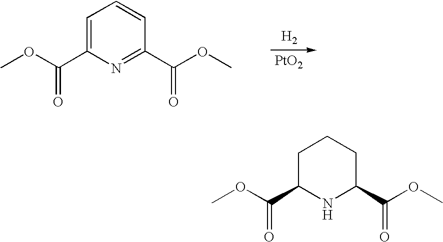

Dimethylpyridine-2,6-dicarboxylate (15 g, 77 mmol) was dissolved in MeOH (150 mL) and HCl(aq) (77 mL, 1M). The reaction vessel was evacuated and backfilled with hydrogen gas, and stirred for 5 days under balloon of hydrogen. When the reaction was complete, the mixture was filtered and concentrated, then dissolved in DCM and washed with Na₂CO₃ (aq). The organic phase was dried, filtered and concentrated to yield the title compound (13.81 g, 89%). ¹H NMR (300 MHz, CDCl₃): δ (ppm) 1.43 (m, 3H); 2.01 (m, 3H); 3.39 (dd, 2H), 3.75 (s, 6H)

### ii) (±)-Methyl (6R,9aS)-1,4-dioxooctahydro-2H-pyrido[1,2-a]pyrazine-6-carboxylate

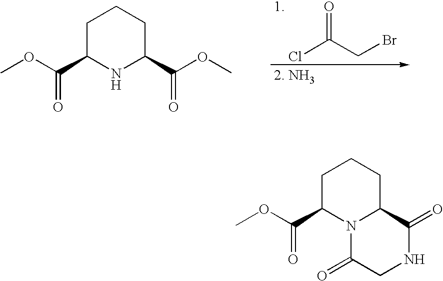

Dimethyl (2R,6S)-piperidine-2,6-dicarboxylate (7 g, 34.8 mmol), and Na₂CO₃ (7.37 g, 69.5 mmol) were added to a round-bottom flask and dissolved in acetonitrile (50 mL and THF (25 mL). The reaction was cooled to 0° C. and bromoacetyl chloride (6.56 g, 41.7 mmol) was added dropwise. The reaction was stirred until the starting material was no longer observed. The solvent was removed in vacuo, and the residue was dissolved in MeOH (40 mL). The solution was cooled to 0 C and concentrated ammonia (20 mL) was added. When the intermediate was consumed, the solvent was removed, and the residue was dissolved in DCM and washed with water. The aqueous phase was extracted again with ethyl acetate and added to the DCM. The organic phase was dried filtered and concentrated, then purified by column chromatography to yield the title compound (6.5 g, 83%). ¹H NMR (300 MHz, CDCl₃): δ (ppm) 1.66 (m, 3H); 1.93 (m, 3H); 3.75 (s, 3H); 4.04 (m, 4H); 7.15 (s, broad, 1H).

### iii) (±)-(6R,9aS)-Octahydro-2H-pyrido[1,2-a]pyrazin-6-ylmethanol

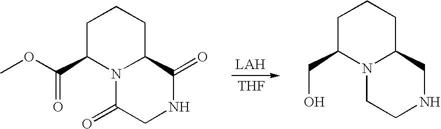

LAH (5.45 g, 143 mmol) was added to a three-neck round bottom flask which was purged with argon. THF (250 mL) was added and cooled to 0 C. (±)-Methyl (6R,9aS)-1,4-dioxooctahydro-2H-pyrido[1,2-a]pyrazine-6-carboxylate (6.5 g, 28.7 mmol) was added as a solid, and the reaction was stirred at 40° C. overnight. The reaction was then cooled to 0° C. and quenched with water, slowly. The mixture was filtered through Celite® and washed with ether and ethyl acetate. The filtrate was evaporated to yield the title compound in quantitative yield. ¹H NMR (300 MHz, CDCl₃): δ (ppm) 1.15 (m, 1H); 1.42 (m, 1H); 1.66 (m, 2H); 1.71 (m, 1H); 2.02-2.07 (m, 4H); 2.53 (dd, 1H); 2.85 (t, 2H); 2.99 (m, 2H); 3.12 (m, 1H); 3.36 (dd, 1H); 3.88 (dd, 1H).

### iv) (±)-Tert-butyl (6R,9aS)-6-(hydroxymethyl)octahydro-2H-pyrido[1,2-a]pyrazine-2-carboxylate

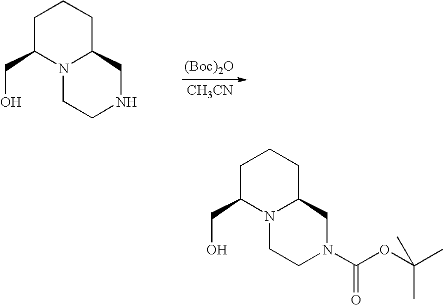

To a solution of (±)-(6R,9aS)-octahydro-2H-pyrido[1,2-a]pyrazin-6-ylmethanol (5.5 g, 32.3 mmol) in DCM (40 mL) was added (Boc)₂O (7 g, 32.3 mmol) at 0° C. The mixture was stirred at 0° C. for 1 h. To the resulting mixture was added NaHCO₃ aq (sat. 200 mL) and extracted with DCM. The combined extract was dried and the solvent was removed with rotary evaporator to give residue which was purified on silica gel column to afford the title compound (4.4 g, 50%). ¹H NMR (300 MHz, CDCl₃): δ (ppm) 1.40 (m, 1H), 1.46 (s, 9H), 1.55-1.8 (m, 5H), 2.05 (s, 3H), 2.5 (broad, 2H), 2.88 (broad, 1H), 3.08 (broad d, 1H), 3.38 (broad d, 1H), 4.14 (m, 3H).

### Example 8.1

### (±)-3-[(6R,8aS)-6-(hydroxymethyl)hexahydropyrrolo[1,2-a]pyrazin-2(1H)-yl]pyrazine-2-carbonitrile

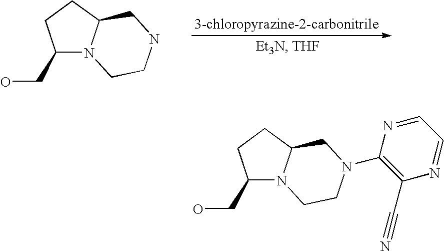

A mixture of (±)-(6R,8aS)-octahydropyrrolo[1,2-a]pyrazin-6-ylmethanol (1.05 g, crude), 3-chloropyrazine-2-carbonitrile (860 mg, 6.2 mmol), Et₃N (1.5 mL) and THF (10 mL) was stirred at 80° C. overnight. After concentration, the crude product was purified on a silica gel column to afford pure product (1.03 g, 77%). ¹H NMR (300 MHz, CDCl₃): δ (ppm) 1.45 (m, 1H), 1.85 (m, 3H), 2.41 (m, 3H), 2.63 (m, 1H), 2.92 (dd, 1H), 3.18 (m, 2H), 3.53 (t, 1H), 3.79 (dd, 1H), 4.61 (m, 2H), 8.03 (s, 1H), 8.27 (s, 1H).

In a similar manner the following compounds were synthesized:

| Example | Structure | Name | Yield |
|---|---|---|---|
| 8.2 | 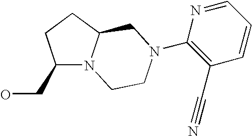 | (±)-2-[(6R,8aS)-6-(hydroxymethyl)hexahydropyrrolo[1,2-a]pyrazin-2(1H)-yl] nicotinonitrile | 60% |
| NMR | 1.43 (m, 1 H), 1.88 (m, 3 H), 2.41 (m, 3 H), 2.48 (m, 1 H), 2.86 (dd, 1 H), 3.14 (m, 2 H), | | |
| | 3.56 (broad d, 1 H), 3.79 (dd, 1 H), 4.49 (m, 2 H), 6.78 (dd, 1 H), 7.79 (dd, 1 H), 8.36 (dd, | | |
| | 1 H). | | |
| 8.3 | 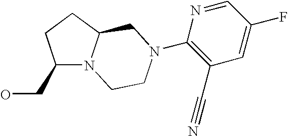 | (±)-5-fluoro-2-[(6R,8aS)-6-(hydroxymethyl)hexahydropyrrolo[1,2-a]pyrazin-2(1H)-yl]nicotinonitrile | 50% |

| | |
|---|---|
| NMR | 1.45 (m, 1 H), 1.88 (m, 3 H), 2.41-2.48 (m, 4 H), 2.85 (dd, 1 H), 3.10 (m, 2 H), 3.54 |
| | (broad d, 1 H), 3.79 (dd, 1 H), 4.26 (m, 2 H), 7.54 (dd, 1 H), 8.26 (dd, 1 H). |

In a similar manner the following compounds were synthesized at 35° C. overnight:

| Example | Structure | Name | Yield |
|---|---|---|---|
| 8.4 | 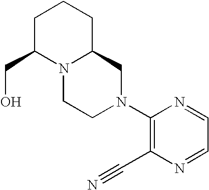 | (±)-3-[(6R,9aS)-6-(hydroxymethyl)octahydro-2H-pyrido[1,2-a]pyrazin-2-yl]pyrazine-2-carbonitrile | 81% |
| NMR | 1.40 (m, 1 H); 1.69 (m, 4 H); 1.82 (m, 1 H); 2.21-2.32 (m, 4 H); 2.93 (dd, 1 H); 3.28 | | |
| | (m, 2 H); 3.35 (d, 1 H); 3.96 (dd, 1 H); 4.34 (d, 1 H); 4.48 (d, 1 H); 8.02 (d, 1 H); | | |
| | 8.26 (d, 1 H). | | |
| 8.5 | 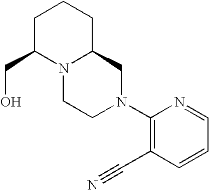 | (±)-2-[(6R,9aS)-6-(hydroxymethyl)octahydro-2H-pyrido[1,2-a]pyrazin-2-yl]nicotinonitrile | 32% |
| NMR | 1.19-1.43 (m, 2 H); 1.64-1.72 (m, 3 H); 1.80 (m, 3 H); 2.19 (m, 1 H); 2.24 (m, 1 H); | | |
| | 2.34 (m, 1 H); 2.88 (m, 1 H); 3.20 (t, 1 H); 3.25 (d, 1 H); 3.39 (d, 1 H); 3.96 (dd, 1 H); | | |
| | 4.21 (d, 1 H); 4.37 (m, 1 H); 6.76 (dd, 1 H); 7.78 (dd, 1 H); 8.35 (dd, 1 H). | | |
| 8.6 | 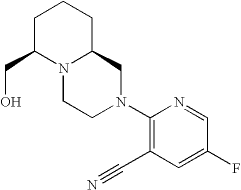 | (±)-5-fluoro-2-[(6R,9aS)-6-(hydroxymethyl)octahydro-2H-pyrido[1,2-a]pyrazin-2-yl]nicotinonitrile | 40% |

| | |
|---|---|
| NMR | 1.39 (m, 1 H); 1.62 (m, 3 H); 1.78 (m, 1 H); 2.24 (m, 3 H); 2.79 (m, 2 H); 3.11 (t, 1 H); |
| | 3.20 (d, 1 H); 3.40 (dd, 1 H); 3.85 (d, 1 H); 3.94 (d, 1 H); 4.04 (m, 1 H); 7.50 (m, 1 H); |
| | 8.19 (m, 1 H). |

### Example 9.1

### (±)-Tert-butyl (6R,8aS)-6-ethynylhexahydropyrrolo[1,2-a]pyrazine-2(1H)-carboxylate

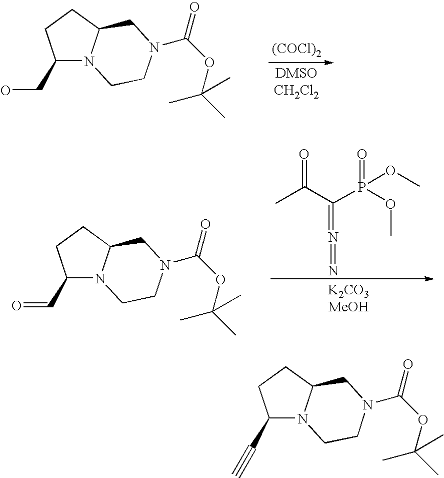

To a solution of oxalyl chloride (2M, 3.3 mL, 6.6 mmol) in DCM (12 mL) was added DMSO (0.71 mL, 10 mmol) at −78° C. After stirring 10 min a solution of (±)-tert-butyl (6R,9aS)-6-(hydroxymethyl)octahydro-2H-pyrido[1,2-a]pyrazine-2-carboxylate (850 mg, 3.3 mmol) in DCM (6 mL) was added. The reaction mixture was stirred at −78° C. for 1 h. Et₃N (2 mL) was added and the resulting mixture was stirred at RT for 30 min, then poured into DCM (30 mL)/NH₃—H₂O (10%, 10 mL). The organic phase was separated, dried over Anhydrous sodium sulphate and concentrated to give crude aldehyde. To the aldehyde was added MeOH (30 mL), K₂CO₃ and dimethyl (1-diazo-2-oxopropyl)phosphonate (768 mg, 4 mmol) at RT. After stirring at RT for 50 min, the resulting mixture was concentrated. The residue was dissolved with ethyl acetate and filtered. After removal of the solvent, flash chromatography on silica gel afforded pure acetylene (557 mg, 64%). ¹H NMR 300 MHz, (CDCl₃) δ (ppm) 1.48 (s, 9H), 1.55 (m, 1H), 1.66-2.2 (m, 5H), 2.34 (s, 1H), 2.63 (broad, 1H), 2.90 (broad t, 2H), 3.25 (broad d, 1H), 4.16 (broad, 2H). In a similar manner the following compounds were synthesized:

| Example | Structure | Name | Yield |
|---|---|---|---|
| 9.2 | 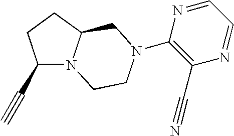 | (±)-3-[(6R,8aS)-6-ethynylhexahydropyrrolo[1,2-a]pyrazin-2(1H)-yl]pyrazine-2-carbonitrile | 53% |
| NMR | 1.58 (m, 1 H), 1.92 (m, 2 H), 2.23 (m, 3 H), 2.35 (s, 1 H), 2.95 (m, 2 H), 3.33 (m, 2 H), | | |
| | 4.53 (m, 2 H), 7.98 (s, 1 H), 8.23 (s, 1 H). | | |
| 9.3 | 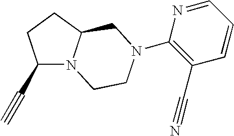 | (±)-2-[(6R,8aS)-6-ethynylhexahydropyrrolo[1,2-a]pyrazin-2(1H)-yl]nicotinonitrile | 54% |
| NMR | 1.65 (m, 1 H), 1.88-2.31 (m, 5 H), 2.36 (s, 1 H), 2.96 (m, 2 H), 3.28-3.44 (m, 2 H), 4.47 | | |
| | (m, 2 H), 6.76 (dd, 1 H), 7.76 (dd, 1 H), 8.35 (dd, 1 H). | | |
| 9.4 | 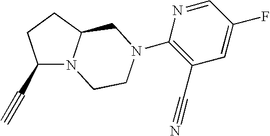 | (±)-2-[(6R,8aS)-6-ethynylhexahydropyrrolo[1,2-a]pyrazin-2(1H)-yl]-5-fluoronicotinonitrile | 55% |
| NMR | 1.57 (m, 1 H), 1.82-2.32 (m, 5 H), 2.37 (s, 1 H), 2.93 (m, 2 H), 3.22-3.41 (m, 2 H), 4.28 | | |
| | (m, 2 H), 7.54 (dd, 1 H), 8.26 (d, 1 H). | | |
| 9.5 | 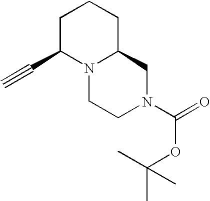 | (±)-tert-butyl (6R,9aS)-6-ethynyloctahydro-2H-pyrido[1,2-a]pyrazine-2-carboxylate | 77% |
| NMR | 1.28 (m, 2 H), 1.47 (s, 9 H), 1.61-2.06 (m, 6 H), 2.35 (s, 1 H), 2.61 (broad, 1 H), 2.78 (d, | | |
| | 1H) 2.98 (broad, 2 H), 3.55 (d, 1 H), 3.8-4.2 (broad 2 H). | | |
| 9.6 | 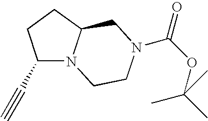 | tert-butyl (6S,8aS)-6-ethynylhexahydropyrrolo[1,2-a]pyrazine-2(1H)-carboxylate | 50% |
| NMR | 1.45 (m, 1 H), 1.47 (s, 9 H), 1.88-1.94 (m, 2 H), 2.18 (m, 1 H), 2.34 (s, 1 H), | | |
| | 2.44-2.81 (m, 5 H), 3.95 (broad d, 1 H), 4.13 (broad, 2 H). | | |
| 9.7 | 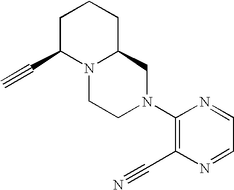 | (±)-3-[(6R,9aS)-6-ethynyloctahydro-2H-pyrido[1,2-a]pyrazin-2-yl]pyrazine-2-carbonitrile | 40% |
| NMR | 1.33 (m, 2 H); 1.70 (m, 1 H); 1.76 (m, 2 H); 1.97 (m, 1 H); 2.09-2.18 (m, 2 H); 2.34 (d, | | |
| | 1 H); 2.80 (d, 1 H); 2.89 (t, 1 H); 3.26 (td, 1 H); 3.64 (d, 1 H); 4.27 (d, 1 H); 4.44 (d, 1 H); | | |
| | 7.94 (d, 1 H); 8.20 (d, 1 H). | | |
| 9.8 | 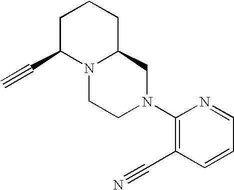 | (±)-2-[(6R,9aS)-6-ethynyloctahydro-2H-pyrido[1,2-a]pyrazin-2-yl]nicotinonitrile | 38% |
| NMR | 1.26 (m, 2 H); 1.55 (m, 1 H); 1.70 (m, 2 H); 1.95 (m, 1 H); 2.07-2.16 (m, 2 H); 2.31 (d, | | |
| | 1 H); 2.79 (m, 2 H); 3.16 (td, 1 H); 3.57 (d, 1 H); 4.11 (d, 1 H); 4.32 (d, 1 H); | | |
| | 6.67 (dd, 1 H); 7.68 (dd, 1 H); 8.25 (dd, 1 H). | | |
| 9.9 | 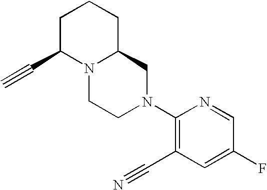 | (±)-2-[(6R,9aS)-6-ethynyloctahydro-2H-pyrido[1,2-a]pyrazin-2-yl]-5-fluoronicotinonitrile | 61% |

| | |
|---|---|
| NMR | 1.28 (m, 2 H); 1.56 (m, 1 H); 1.70 (m, 2 H); 1.95 (m, 1 H); 2.10-2.17 (m, 2 H); 2.30 (d, |
| | 1 H); 2.76 (m, 2 H); 3.14 (td, 1 H); 3.57 (d, 1 H); 3.94 (d, 1 H); 4.09 (d, |
| | 1 H); 7.46 (dd, 1 H); 8.15 (d, 1 H). |

### Example 10.1

### (±)-(6R,8aS)-6-[(3-chlorophenyl)ethynyl]octahydropyrrolo[1,2-a]pyrazine

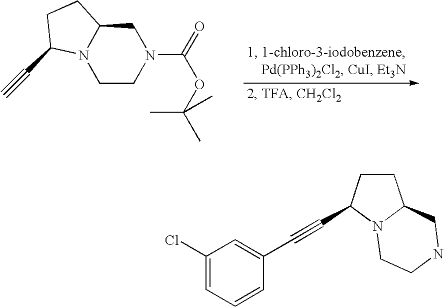

A mixture of (±)-tert-butyl (6S,8aR)-6-ethynylhexahydropyrrolo[1,2-a]pyrazine-2(1H)-carboxylate (557 mg, 2.1 mmol), 3-iodo-chlorobenzene (952 mg, 4 mmol), Pd(PPh₃)₂Cl₂ (84 mg, 0.12 mmol), CuI (45 mg, 0.24 mmol) and Et₃N (4 mL) was stirred at RT overnight. After removal of amine with air flow, the residue was purified on silica gel column. The resulting phenyl acetylene in DCM (2 mL)/TFA (1 mL) was stirred at RT for 2 h. DCM and excess TFA were removed in vacuo. The residue was diluted with ethyl acetate and washed with aqueous Na₂CO₃. The organic phase was dried over Anhydrous sodium sulphate. After filtration, the solvent was removed in vacuo to afford the title compound (367 mg, 63%). ¹H NMR (300 MHz, CDCl₃): δ (ppm) 1.55 (m, 1H), 1.85-2.25 (m, 5H), 2.69 (dd, 1H), 2.96 (dd, 1H), 3.14-3.55 (m, 4H), 7.26 (m, 3H), 7.45 (s, 1H).

In a similar manner the following compounds were synthesized:

| Example | Structure | Name | Yield |
|---|---|---|---|
| 10.2 | 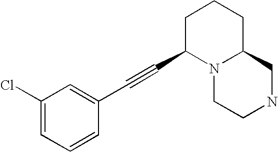 | (±)-(6R,9aS)-6-[(3-chlorophenyl)ethynyl]octahydro-2H-pyrido[1,2-a]pyrazine | 86% |
| NMR | 1.22-2.18 (m, 8 H), 2.63 (dd, 1 H), 2.90 (dd, 1 H), 3.03 (m, 3 H), 3.60 (dt, 1 H), 7.27 (m, | | |
| | 3 H), 7.42 (s, 1 H). | | |
| 10.3 | 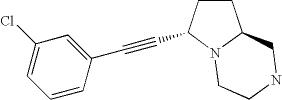 | (6S,8aS)-6-[(3-chlorophenyl)ethynyl]octahydropyrrolo[1,2-a]pyrazine | 91% |
| NMR | 1.70 (m, 1 H), 2.14-2.46 (m, 3 H), 2.12-3.68 (m, 7 H), 4.49 (m, 1 H), 7.29 (m, 3 H), 7.43 | | |
| | (s, 1 H). | | |
| 10.4 | 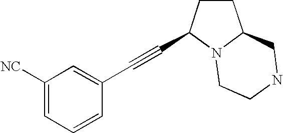 | (±)-3-[(6R,8aS)-octahydropyrrolo[1,2-a]pyrazin-6-ylethynyl]benzonitrile | |

| | |
|---|---|
| NMR | 1.58 (m, 1 H), 1.88-2.23 (m, 5 H), 2.71 (dd, 1 H), 3.06 (dd, 1 H), 3.22-3.48 (m, 4 H), 7.44 |
| | (dd, 3 H), 7.59 (d, 1 H), 7.65 (d, 1 H), 7.73 (s, 1 H). |

### Example 11

### (±)-(9aS)-6-[(E)-2-(3-chlorophenyl)vinyl]octahydro-2H-pyrido[1,2-a]pyrazine

### i) (3-chlorobenzyl)(triphenyl)phosphonium bromide

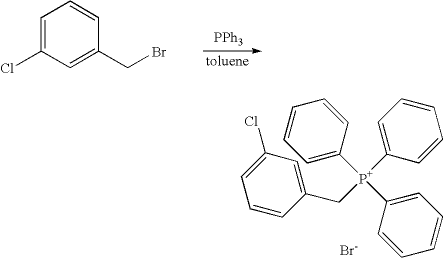

A mixture of 1-(bromomethyl)-3-chlorobenzene (5.5 mL, 42 mmol) and triphenylphosphine (7.8 g, 30 mmol) in toluene (80 mL) was heated at reflux for 6 h. After cooling to RT, the solid was collected by filtration and rinsed with benzene and hexane to yield the title compound (13.7 g, 98%).

### ii) (±)-Tert-butyl (9aS)-6-[(E)-2-(3-chlorophenyl)vinyl]octahydro-2H-pyrido[1,2-a]pyrazine-2-carboxylate

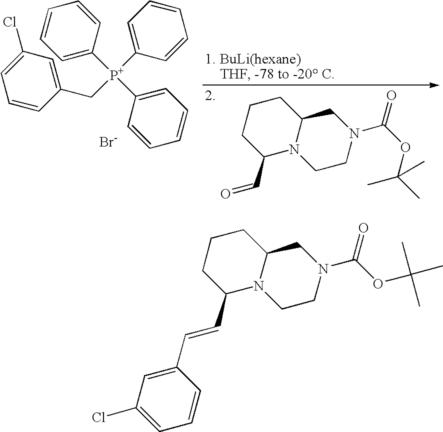

nBuLi (1.6 mL, 1.6M in hexane, 2.6 mmol) was added to a suspension of (3-chlorobenzyl)(triphenyl)phosphonium bromide (1.1 g, 2.4 mmol) in THF (13 mL) at −78° C. The mixture was warmed to −20° C. over 3 min and a solution of (±)-tert-butyl (9aS)-6-formyloctahydro-2H-pyrido[1,2-a]pyrazine-2-carboxylate (1.9 mmol generated from 512 mg of alcohol via Swern oxidation as in example above) was added. The resulting mixture was allowed to warm to RT overnight. The mixture was partitioned between ethyl acetate and water. After the organic phase was dried and concentrated in vacuo, flash column chromatography yielded the title compound (347 mg, 48%).

### iii) (±)-(9aS)-6-[(E)-2-(3-chlorophenyl)vinyl]octahydro-2H-pyrido[1,2-a]pyrazine

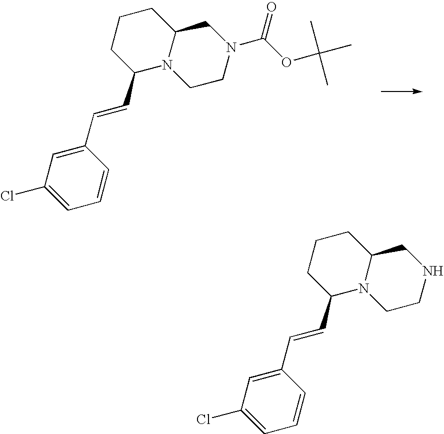

To a mixture of (±)-tert-butyl (9aS)-6-[(E)-2-(3-chlorophenyl)vinyl]octahydro-2H-pyrido[1,2-a]pyrazine-2-carboxylate (347 mg, 0.96 mmol) and DCM (3 mL) was added TFA (2 mL) at 0° C. The mixture was stirred at RT for 2 h. After removal of DCM and TFA, the residue was diluted with DCM and washed with Na₂CO₃ aq. Organic solution was dried over Anhydrous sodium sulphate and the solvent was removed to afford the title product (218 mg, 85%) which was used without further purification.

### Example 12.1

### (±)-Methyl (6R,8aS)-6-[(3-chlorophenyl)ethynyl]hexahydropyrrolo[1,2-a]pyrazine-2(1H)-carboxylate

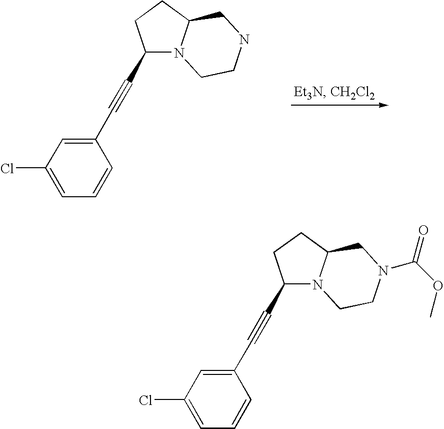

To a mixture of (±)-(6R,8aS)-6-[(3-chlorophenyl)ethynyl]octahydropyrrolo[1,2-a]pyrazine (40 mg, 0.15 mmol) Et₃N (40 mg, 0.4 mmol) and DCM (1 mL) was added methyl chloroformate (30 mg, 0.3 mmol) at −78° C. The resulting mixture was stirred at RT for 15 min, then washed with NaHCO₃ aq (sat.) and subjected to silica gel column to afford product (40 mg, 84%). ¹H NMR (300 MHz, CDCl₃): δ (ppm) 1.55 (m, 1H), 1.85-2.25 (m, 5H), 2.69 (broad, 1H), 3.05 (broad, 1H), 3.16 (dd, 1H), 3.24 (broad d, 1H), 3.73 (s, 1H), 4.2 (broad, 2H), 7.28 (m, 3H), 7.45 (s, 1H).

In a similar manner the following compounds were synthesized:

| Example | Structure | Name | Yield |
|---|---|---|---|
| 12.2 | 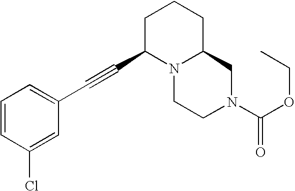 | (±)-ethyl (6R,9aS)-6-[(3-chlorophenyl)ethynyl]octahydro-2H-pyrido[1,2-a]pyrazine-2-carboxylate | 81% |
| NMR | 1.39 (t, 3 H), 1.40 (m, 2 H), 1.59 (broad d, 1 H), 1.80-2.12 (m, 5 H), 2.69 (broad, 1 H), 3.03 | | |
| | (dd, 1 H), 3.06 (broad, 1 H), 3.55 (broad d, 1 H), 3.93 (broad, 2 H), 4.15 (q, 2 H), 7.28 (m, | | |
| | 3 H), 7.42 (s, 1 H). | | |
| 12.3 | 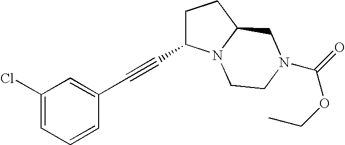 | ethyl (6S,8aS)-6-[(3-chloro-phenyl)ethynyl]hexahydropyrrolo[1,2-a]pyrazine-2(1H)-carboxylate | 70% |
| NMR | 1.28 (t, 3 H), 1.46 (m, 1 H), 2.0 (m, 2 H), 2.26 (m, 1 H), 2.60 (m, 3 H), 2.92 (m, 2 H), 4.18 | | |
| | (q, 2 H), 4.20 (broad, 3 H), 7.27 (m, 3 H), 7.40 (s, 1 H). | | |
| 12.4 | 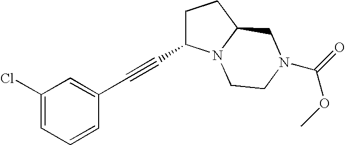 | methyl (6S,8aS)-6-[(3-chloro-phenyl)ethynyl]hexahydropyrrolo[1,2-a]pyrazine-2(1H)-carboxylate | 83% |
| NMR | 1.45 (m, 1 H), 1.97 (m, 2 H), 2.26 (m, 1 H), 2.53-2.92 (m, 5 H), 3.72 (s, 1 H), 4.15 (broad, | | |
| | 3 H), 7.28 (m, 3 H), 7.40 (s, 1 H). | | |
| 12.5 | 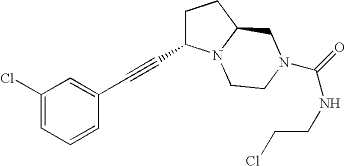 | (6S,8aS)-N-(2-chloroethyl)-6-[(3-chlorophenyl)ethynyl]hexahydro-pyrrolo[1,2-a]pyrazine-2(1H)-carboxamide | 13% |
| NMR | 1.42 (m, 1 H), 2.02 (m, 2 H), 2.28 (m, 1 H), 2.58-3.03 (m, 5 H), 3.60-3.70 (m, 4 H), 3.95 | | |
| | (broad d, 1 H), 4.09 (d, 1 H), 4.20 (d, 1 H), 5.32 (t, 1 H), 7.29 (m, 3 H), 7.41 (s, 1 H). | | |
| 12.6 | 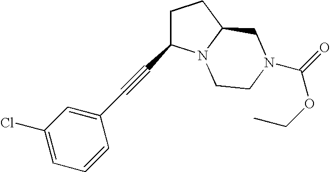 | (±)-ethyl (6R,8aS)-6-[(3-chloro-phenyl)ethynyl]hexahydropyrrolo[1,2-a]pyrazine-2(1H)-carboxylate | 76% |
| NMR | 1.29 (t, 3 H), 1.38 (m, 1 H), 1.80-2.26 (m, 5 H), 2.69 (broad, 1 H), 3.01 (dd, 1 H), 3.19 (dd, | | |
| | 1 H), 3.31 (d, 1 H), 4.15 (q, 2 H), 4.20 (broad, 2 H), 7.28 (m, 3 H), 7.44 (s, 1 H). | | |
| 12.7 | 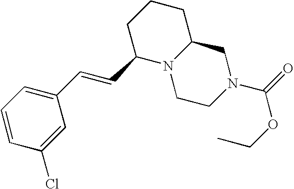 | (±)-ethyl (6R,9aS)-6-[(E)-2-(3-chlorophenyl)vinyl]octahydro-2H-pyrido[1,2-a]pyrazine-2-carboxylate | 99% |

| | |
|---|---|
| NMR | 1.25 (t, 3 H), 1.38-2.01 (m, 8 H), 2.64 (m, 2 H), 3.01 (broad, 2 H), 4.05 (broad, 2 H), 4.15 |
| | (q, 2 H), 6.15 (dd, 1 H), 6.45 (d, 1 H), 7.24 (m, 3 H), 7.36 (s, 1 H). |

### Example 13.1

### (±)-3-[(6R,8aS)-6-[(3-chlorophenyl)ethynyl]hexahydropyrrolo[1,2-a]pyrazin-2(1H)-yl]pyrazine-2-carbonitrile

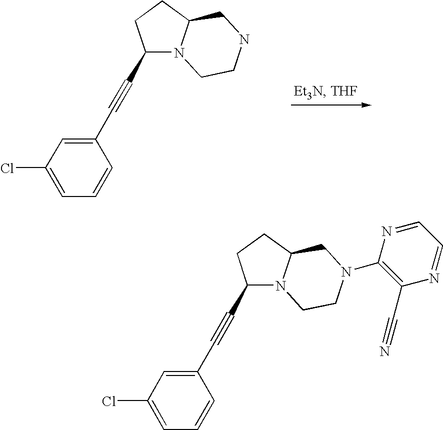

A mixture of (±)-(6R,8aS)-6-[(3-chlorophenyl)ethynyl]octahydropyrrolo[1,2-a]pyrazine (40 mg, 0.15 mmol), 2-chloro-3-cyanopyrazine (30 mg, 0.22 mmol), Et₃N (0.1 mL) and THF (1.5 mL) was stirred at 80° C. for 4 h. The resulting mixture was concentrated and purified on silica gel column to provide product (44 mg, 81%). ¹H NMR (300 MHz, CDCl₃): δ (ppm) 1.62 (m, 1H), 1.85-2.25 (m, 5H), 3.02 (dd, 1H), 3.24-3.48 (m, 3H), 4.61 (dt, 2H), 7.29 (m, 3H), 7.45 (s, 1H), 8.02 (d, 1H), 8.27 (d, 1H).

In a similar manner the following compounds were synthesized:

| Example | Structure | Name | Yield |
|---|---|---|---|
| 13.2 | 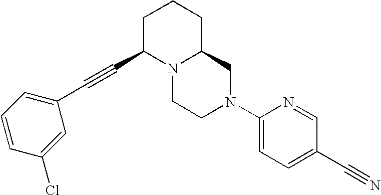 | (±)-6-{(6R,9aS)-6-[(3-chlorophenyl)ethynyl]octahydro-2H-pyrido[1,2-a]pyrazin-2-yl}nicotinonitrile | 51% |
| NMR | 1.43 (m, 2 H), 1.69 (m, 2 H), 1.88 (m, 2 H), 2.09 (m, 2 H), 2.82 (dd, 1 H), 3.03 (dd, 1 H), | | |
| | 3.21 (dt, 1 H), 3.70 (d, 1 H), 4.29 (d, 2 H), 6.6 (d, 1 H), 7.29 (m, 3 H), 7.43 (s, 1 H), 7.63 (d, | | |
| | 1 H), 8.42 (s, 1 H). | | |
| 13.3 | 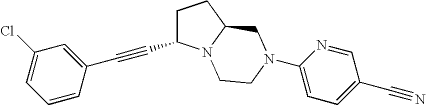 | 6-[(6S,8aS)-6-[(3-chlorophenyl)ethynyl]hexahydro-pyrrolo[1,2-a]pyrazin-2(1H)-yl]nicotinonitrile | 40% |
| NMR | 1.53 (m, 1 H), 2.06 (m, 2 H), 2.35 (m, 1 H), 2.74 (m, 3 H), 3.09 (m, 2 H), 4.23 (d, 1 H), 4.39 | | |
| | (d, 1 H), 4.60 (d, 1 H), 6.6 (d, 1 H), 7.28 (m, 3 H), 7.38 (s, 1 H), 7.62 (d, 1 H), 8.42 (s, 1 H). | | |
| 13.4 | 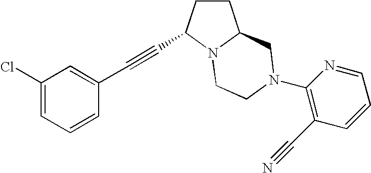 | 2-[(6S,8aS)-6-[(3-chlorophenyl)ethynyl]hexahydropyrrolo[1,2-a]pyrazin-2(1H)-yl]nicotinonitrile | 30% |
| NMR | 1.52 (m, 1 H), 2.05 (m, 2 H), 2.34 (m, 1 H), 2.74 (m, 3 H), 3.05 (m, 1 H), 3.21 (dt, 1 H), | | |
| | 4.24 (d, 1 H), 4.46 (d, 1 H), 4.58 (d, 1 H), 6.75 (dd, 1 H), 7.28 (m, 3 H), 7.39 (s, 1 H), 7.78 | | |
| | (dd, 1 H), 8.42 (d, 1 H). | | |
| 13.5 | 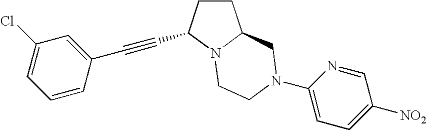 | (6S,8aS)-6-[(3-chlorophenyl)ethynyl]-2-(5-nitropyridin-2-yl)octahydropyrrolo[1,2-a]pyrazine | 50% |
| NMR | 1.57 (m, 1 H), 2.05 (m, 2 H), 2.35 (m, 1 H), 2.64-3.21 (m, 5 H), 4.26 (d, 1 H), 4.50 (d, 1 H), | | |
| | 4.71 (d, 1 H), 6.62 (d, 1 H), 7.26 (m, 3 H), 7.38 (s, 1 H), 8.21 (dd, 1 H), 9.06 (d, 1 H). | | |
| 13.6 | 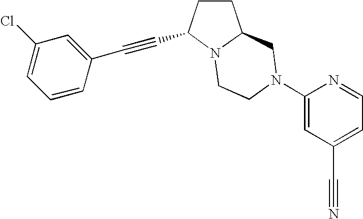 | 2-[(6S,8aS)-6-[(3-chlorophenyl)ethynyl]hexahydropyrrolo[1,2-a]pyrazin-2(1H)-yl]isonicotinonitrile | 32% |
| NMR | 1.57 (m, 1 H), 2.06 (m, 2 H), 2.33 (m, 1 H), 2.68-3.21 (m, 5 H), 4.23-4.50 (m, 3 H), 6.75 (d, | | |
| | 1 H), 6.86 (s, 1 H), 7.26 (m, 3 H), 7.38 (s, 1 H), 8.30 (dd, 1 H). | | |
| 13.7 | 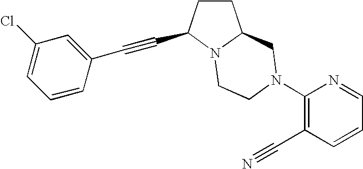 | (±)-2-[(6R,8aS)-6-[(3-chlorophenyl)ethynyl]hexahydropyrrolo[1,2-a]pyrazin-2(1H)-yl]nicotinonitrile | 43% |
| NMR | 1.67 (m, 1 H), 1.82-2.4 (m, 5 H), 2.98 (dd, 1 H), 3.26 (m, 2 H), 3.45 (d, 1 H), 4.50 (m, 2 H), | | |
| | 6.76 (dd, 1 H), 7.28 (m, 3 H), 7.45 (s, 1 H), 7.78 (dd, 1 H), 8.35 (dd, 1 H). | | |
| 13.8 | 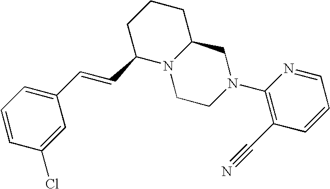 | (±)-2-{(6R,9aS)-6-[(E)-2-(3-chloro-phenyl)vinyl]octahydro-2H-pyrido[1,2-a]pyrazin-2-yl}nicotinonitrile | 12% |
| NMR | 1.38-1.82 (m, 6 H), 2.20 (m, 2 H), 2.72 (t, 1 H), 2.91 (t, 1 H), 3.17 (t, 2 H), 4.27 (m, 2 H), | | |
| | 6.19 (dd, 1 H), 6.60 (d, 1 H), 6.76 (dd, 1 H), 7.25 (m, 3 H), 7.37 (s, 1 H), 7.78 dd, 1 H), | | |
| | 8.35 (dd, 1 H). | | |
| 13.9 | 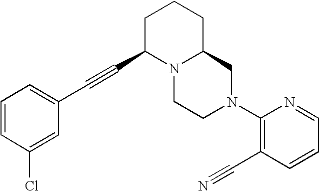 | (±)-2-{(6R,9aS)-6-[(3-chlorophenyl)ethynyl]octahydro-2H-pyrido[1,2-a]pyrazin-2-yl}nicotinonitrile | 50% |
| NMR | 1.26-2.35 (m, 8 H), 2.93 (dd, 1 H), 3.08 (dd, 1 H), 3.30 (m, 1 H), 3.71 (m, 1 H), 4.24-4.45 | | |
| | (m, 2 H), 6.76 (dd, 1 H), 7.28 (m, 3 H), 7.43 (s, 1 H), 7.78 (dd, 1 H), 8.35 (dd, 1 H). | | |
| 13.10 | 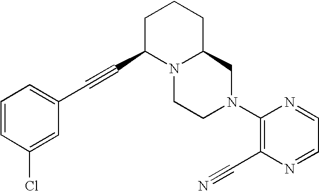 | (±)-3-{(6R,9aS)-6-[(3-chlorophenyl)ethynyl]octahydro-2H-pyrido[1,2-a]pyrazin-2-yl}pyrazine-2-carbonitrile | 60% |
| NMR | 1.26 (m, 2 H), 1.73-2.31 (m, 6 H), 2.98 (dd, 1 H), 3.07 (m, 1 H), 3.35 (dt, 1 H), 3.74 (d, | | |
| | 1 H), 4.46 (d, 1 H), 4.52 (d, 1 H), 7.28 (m, 3 H), 7.43 (s, 1 H), 8.02 (d, 1 H), 8.26 (d, 1 H). | | |
| 13.11 | 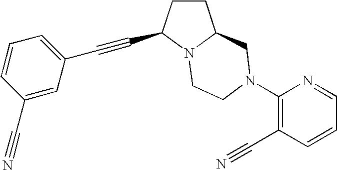 | (±)-2-[(6R,8aS)-6-[(3-cyanophenyl)ethynyl]hexahydropyrrolo[1,2-a]pyrazin-2(1H)-yl]nicotinonitrile | 20% |
| NMR | 1.65 (m, 1 H), 1.83-2.38 (m, 5 H), 2.98 (dd, 1 H), 3.21-3.46 (m, 3 H), 4.50 (m, 2 H), 6.75 | | |
| | (dd, 1 H), 7.43 (dd, 1 H), 7.58 (d, 1 H), 7.73-7.80 (m, 3 H), 8.36 (d, 1 H). | | |
| 13.12 | 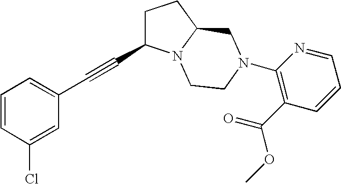 | (±)-methyl 2-[(6R,8aS)-6-[(3-chloro-phenyl)ethynyl]hexahydropyrrolo[1,2-a]pyrazin-2(1H)-yl]nicotinate | 48% |
| NMR | 1.62-2.35 (m, 6 H), 2.95 (dd, 1 H), 3.19-3.41 (m, 3 H), 3.83-4.04 (m, 2 H), 3.91 (s, 3 H), | | |
| | 6.75 (dd, 1 H), 7.26 (m, 3 H), 7.46 (s, 1 H), 7.99 (dd, 1 H), 8.30 (d, 1 H). | | |
| 13.13 | 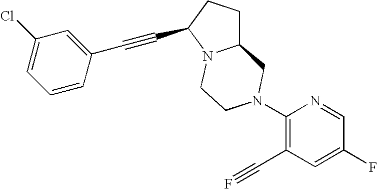 | (±)-2-[(6R,8aS)-6-[(3-chlorophenyl)ethynyl]hexahydropyrrolo[1,2-a]pyrazin-2(1H)-yl]-5-fluoro-nicotinonitrile | 18% |
| NMR | 1.62 (m, 1 H), 1.88-2.38 (m, 5 H), 2.96 (dd, 1 H), 3.21-3.47 (m, 3 H), 4.3 (m, 2 H), 7.24 | | |
| | (m, 3 H), 7.45 (s, 1 H), 7.56 (dd, 1 H), 8.26 (d, 1 H). | | |
| 13.14 | 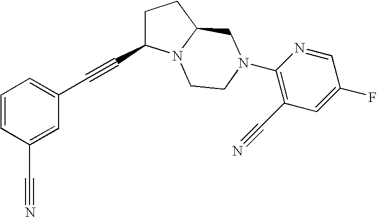 | (±)-2-[(6R,8aS)-6-[(3-cyanophenyl)ethynyl]hexahydropyrrolo[1,2-a]pyrazin-2(1H)-yl]-5-fluoro-nicotinonitrile | 25% |

| | |
|---|---|
| NMR | 1.64 (m, 1 H), 1.90-2.40 (m, 5 H), 2.97 (dd, 1 H), 3.22-3.46 (m, 3 H), 4.3 (m, 2 H), 7.43- |
| | 7.69 (m, 4 H), 7.45 (s, 1 H), 8.38 (d, 1 H). |

### Example 14.1

### (±)-3-[(6R,8aS)-6-[(3-cyanophenyl)ethynyl]hexahydropyrrolo[1,2-a]pyrazin-2(1H)-yl]pyrazine-2-carbonitrile

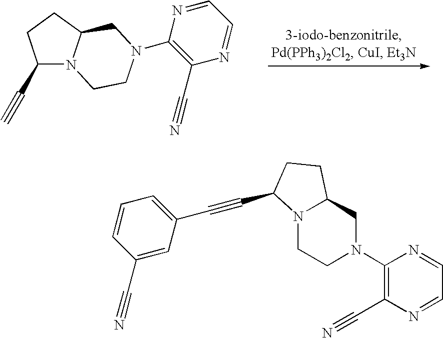

A mixture of (±)-3-[(6R,8aS)-6-ethynylhexahydropyrrolo[1,2-a]pyrazin-2(1H)-yl]pyrazine-2-carbonitrile (40 mg, 0.15 mmol), 3-iodobenzonitrile (68 mg, 0.3 mmol), Pd(PPh₃)₂Cl₂ (8 mg, 0.011 mmol), CuI (4 mg, 0.02 mmol) and Et₃N (0.8 mL) was stirred at RT under argon overnight. The resulting mixture was concentrated and purified on silica gel column to afford the title compound (53 mg, 99%). ¹H NMR 300 MHz, (CDCl₃) δ (ppm) 1.65 (m, 1H), 1.89-2.4 (m, 5H), 3.02 (dd, 1H), 3.23-3.51 (m, 3H), 4.62 (m, 2H), 7.45 (t, 1H), 7.6-7.76 (m, 3H), 8.02 (d, 1H), 8.28 (d, 1H). In a similar manner the following compounds were synthesized:

| Example | Structure | Name | Yield |
|---|---|---|---|
| 14.2 | 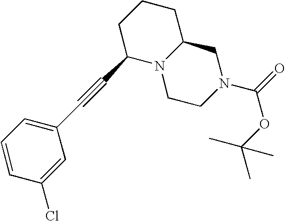 | (±)-tert-butyl (6R,9aS)-6-[(3-chlorophenyl)ethynyl]octahydro-2H-pyrido[1,2-a]pyrazine-2-carboxylate | 86% |
| NMR | 1.38 (m, 2 H), 1.60-2.18 (m, 6 H), 2.65 (broad, 1 H), 3.03 (d, 2 H), 3.6 (d, 1 H), 4.0 (broad, | | |
| | 2 H), 7.28 (m, 3 H), 7.42 (s, 1 H). | | |
| 14.3 | 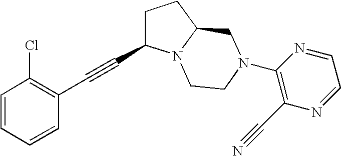 | (±)-3-[(6R,8aS)-6-[(2-chlorophenyl)ethynyl]hexahydropyrrolo[1,2-a]pyrazin-2(1H)-yl]pyrazine-2-carbonitrile | 100% |
| NMR | 1.65 (m, 1 H), 1.82-2.37 (m, 5 H), 3.04 (dd, 1 H), 3.29-3.59 (m, 3 H), 4.62 (m, 2 H), 7.21- | | |
| | 7.52 (m, 4 H), 8.02 (d, 1 H), 8.27 (d, 1 H). | | |
| 14.4 | 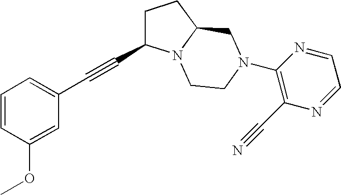 | (±)-3-[(6R,8aS)-6-[(3-methoxy-phenyl)ethynyl]hexahydropyrrolo[1,2-a]pyrazin-2(1H)-yl]pyrazine-2-carbonitrile | 100% |
| NMR | 1.67 (m, 1 H), 1.82-2.34 (m, 5 H), 3.04 (dd, 1 H), 3.22-3.50 (m, 3 H), 3.81 (s, 3 H), 4.62 | | |
| | (m, 2 H), 6.88-7.28 (m, 4 H), 8.03 (d, 1 H), 8.27 (d, 1 H). | | |
| 14.5 | 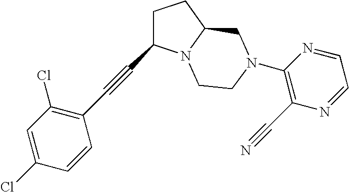 | (±)-3-[(6R,8aS)-6-[(2,4-dichloro-phenyl)ethynyl]hexahydropyrrolo[1,2-a]pyrazin-2(1H)-yl]pyrazine-2-carbonitrile | 93% |
| NMR | 1.67 (m, 1 H), 1.89-2.37 (m, 5 H), 3.02 (dd, 1 H), 3.28-3.55 (m, 3 H), 4.64 (m, 2 H), 7.28 | | |
| | (m, 2 H), 7.48 (s, 1 H), 8.03 (d, 1 H), 8.27 (d, 1 H). | | |
| 14.6 | 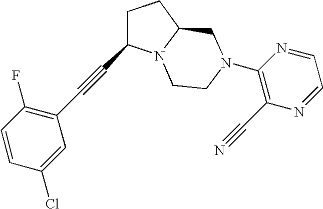 | (±)-3-[(6R,8aS)-6-[(5-chloro-2-fluorophenyl)ethynyl]hexahydropyrrolo[1,2-a]pyrazin-2(1H)-yl]pyrazine-2-carbonitrile | 53% |
| NMR | 1.67 (m, 1 H), 1.95-2.35 (m, 5 H), 3.03 (dd, 1 H), 3.25-3.51 (m, 3 H), 4.60 (m, 2 H), 7.02- | | |
| | 7.45 (m, 3 H), 8.03 (d, 1 H), 8.27 (d, 1 H). | | |
| 14.7 | 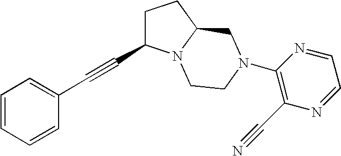 | (±)-3-[(6R,8aS)-6-[(phenylethynyl)hexahydropyrrolo[1,2-a]pyrazin-2(1H)-yl]pyrazine-2-carbonitrile | 100% |
| NMR | 1.69 (m, 1 H), 1.90-2.35 (m, 5 H), 3.05 (dd, 1 H), 3.22-3.54 (m, 3 H), 4.61 (m, 2 H), 7.31- | | |
| | 7.49 (m, 5 H), 8.03 (d, 1 H), 8.27 (d, 1 H). | | |
| 14.8 | 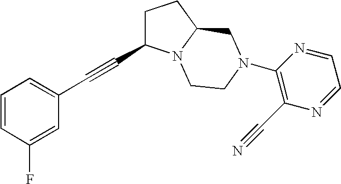 | (±)-3-[(6R,8aS)-6-[(3-fluorophenyl)ethynyl]hexahydropyrrolo[1,2-a]pyrazin-2(1H)-yl]pyrazine-2-carbonitrile | 100% |
| NMR | 1.65 (m, 1 H), 1.90-2.37 (m, 5 H), 3.04 (dd, 1 H), 3.22-3.52 (m, 3 H), 4.61 (m, 2 H), 7.03- | | |
| | 7.30 (m, 4 H), 8.03 (d, 1 H), 8.27 (d, 1 H). | | |
| 14.9 | 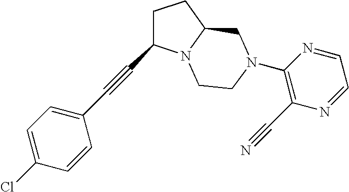 | (±)-3-[(6R,8aS)-6-[(4-chlorophenyl)ethynyl]hexahydropyrrolo[1,2-a]pyrazin-2(1H)-yl]pyrazine-2-carbonitrile | 89% |
| NMR | 1.68 (m, 1 H), 1.93-2.34 (m, 5 H), 3.02 (dd, 1 H), 3.21-3.52 (m, 3 H), 4.60 (m, 2 H), 7.29 | | |
| | (d, 2 H), 7.39 (d, 2 H), 8.03 (d, 1 H), 8.27 (d, 1 H). | | |
| 14.10 | 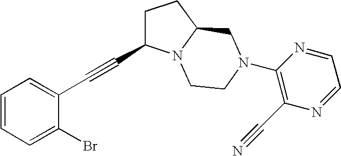 | (±)-3-[(6R,8aS)-6-[(2-bromophenyl)ethynyl]hexahydropyrrolo[1,2-a]pyrazin-2(1H)-yl]pyrazine-2-carbonitrile | 61% |
| NMR | 1.68 (m, 1 H), 1.93-2.37 (m, 5 H), 3.04 (dd, 1 H), 3.29-3.62 (m, 3 H), 4.65 (m, 2 H), 7.18- | | |
| | 7.61 (m, 4 H), 8.03 (d, 1 H), 8.27 (d, 1 H). | | |
| 14.11 | 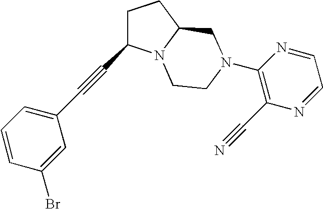 | (±)-3-[(6R,8aS)-6-[(3-bromophenyl)ethynyl]hexahydropyrrolo[1,2-a]pyrazin-2(1H)-yl]pyrazine-2-carbonitrile | 100% |
| NMR | 1.68 (m, 1 H), 1.93-2.34 (m, 5 H), 3.04 (dd, 1 H), 3.22-3.51 (m, 3 H), 4.64 (m, 2 H), | | |
| | 7.16 (dd, 1 H), 7.38 (d, 1 H), 7.45 (d, 1 H), 7.62 (s, 1 H), 8.03 (d, 1 H), 8.27 (d, 1 H). | | |
| 14.12 | 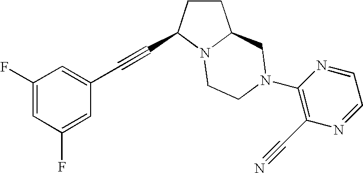 | (±)-3-[(6R,8aS)-6-[(3,5-difluoro-phenyl)ethynyl]hexahydropyrrolo[1,2-a]pyrazin-2(1H)-yl]pyrazine-2-carbonitrile | 85% |
| NMR | 1.66 (m, 1 H), 1.95-2.36 (m, 5 H), 3.02 (dd, 1 H), 3.21-3.49 (m, 3 H), 4.63 (m, 2 H), 6.79 | | |
| | (m, 1 H), 6.98 (m, 2 H), 8.03 (d, 1 H), 8.27 (d, 1 H). | | |
| 14.13 | 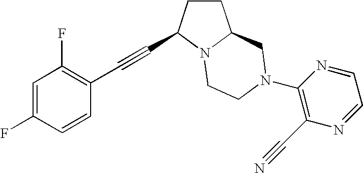 | (±)-3-[(6R,8aS)-6-[(2,4-difluoro-phenyl)ethynyl]hexahydropyrrolo[1,2-a]pyrazin-2(1H)-yl]pyrazine-2-carbonitrile | 62% |
| NMR | 1.67 (m, 1 H), 1.95-2.36 (m, 5 H), 3.02 (dd, 1 H), 3.25-3.52 (m, 3 H), 4.61 (m, 2 H), 6.85 | | |
| | (m, 2 H), 7.42 (m, 1 H), 8.03 (d, 1 H), 8.27 (d, 1 H). | | |
| 14.14 | 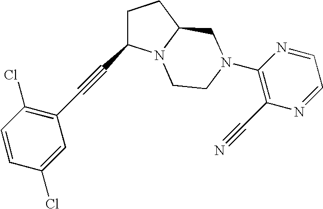 | (±)-3-[(6R,8aS)-6-[(2,5-dichloro-phenyl)ethynyl]hexahydropyrrolo[1,2-a]pyrazin-2(1H)-yl]pyrazine-2-carbonitrile | 100% |
| NMR | 1.65 (m, 1 H), 1.95-2.37 (m, 5 H), 3.02 (dd, 1 H), 3.27-3.55 (m, 3 H), 4.61 (m, 2 H), 7.21 | | |
| | (d, 1 H), 7.34 (d, 1 H), 7.62 (s, 1 H), 8.03 (d, 1 H), 8.27 (d, 1 H). | | |
| 14.15 | 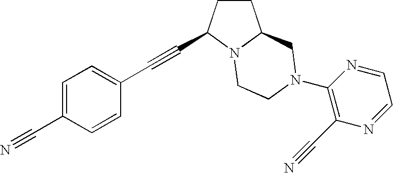 | (±)-3-[(6R,8aS)-6-[(4-cyanophenyl)ethynyl]hexahydropyrrolo[1,2-a]pyrazin-2(1H)-yl]pyrazine-2-carbonitrile | 100% |
| NMR | 1.65 (m, 1 H), 1.95-2.36 (m, 5 H), 3.01 (dd, 1 H), 3.24-3.48 (m, 3 H), 4.61 (m, 2 H), 7.52 | | |
| | (d, 2 H), 7.62 (d, 2 H), 8.03 (d, 1 H), 8.27 (d, 1 H). | | |
| 14.16 | 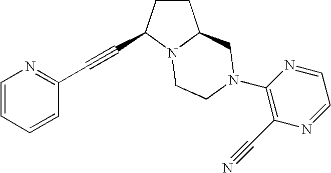 | (±)-3-[(6R,8aS)-6-(pyridin-2-yl-ethynyl)hexahydropyrrolo[1,2-a]pyrazin-2(1H)-yl]pyrazine-2-carbonitrile | 74% |
| NMR | 1.65 (m, 1 H), 1.95-2.34 (m, 5 H), 3.00 (dd, 1 H), 3.26-3.54 (m, 3 H), 4.59 (m, 2 H), 7.22 | | |
| | (m, 1 H), 7.44 (d, 1 H), 7.63 (m, 1 H), 8.01 (d, 1 H), 8.25 (d, 1 H), 8.56 (d, 1 H). | | |
| 14.17 | 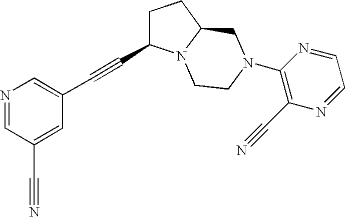 | (±)-3-[(6R,8aS)-6-[(5-cyanopyridin-3-yl)ethynyl]hexahydropyrrolo[1,2-a]pyrazin-2(1H)-yl]pyrazine-2-carbonitrile | 92% |
| NMR | 1.65 (m, 1 H), 1.95-2.34 (m, 5 H), 3.00 (dd, 1 H), 3.26-3.48 (m, 3 H), 4.61 (m, 2 H), 8.01 | | |
| | (s, 1 H), 8.05 (d, 1 H), 8.27 (d, 1 H), 8.80 (s, 1 H), 8.86 (s, 1 H). | | |
| 14.18 | 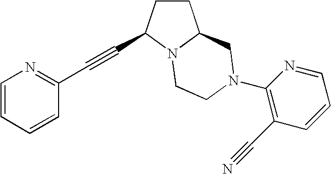 | (±)-2-[(6R,8aS)-6-(pyridin-2-yl-ethynyl)hexahydropyrrolo[1,2-a]pyrazin-2(1H)-yl]nicotinonitrile | 26% |
| NMR | 1.65 (m, 1 H), 1.90-2.38 (m, 5 H), 2.98 (dd, 1 H), 3.28-3.53 (m, 3 H), 4.49 (m, 2 H), 6.76 | | |
| | (dd, 1 H), 7.25 (dd, 1 H), 7.46 (d, 1 H), 7.63 (dd, 1 H), 7.78 (d, 1 H), 8.35 (dd, 1 H), 8.37 (d, | | |
| | 1 H). | | |
| 14.19 | 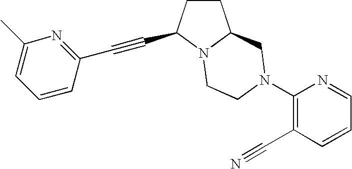 | (±)-2-[(6R,8aS)-6-[(6-methylpyridin-2-yl)ethynyl]hexahydropyrrolo[1,2-a]pyrazin-2(1H)-yl]nicotinonitrile | 33% |
| NMR | 1.63 (m, 1 H), 1.88-2.40 (m, 5 H), 2.56 (s, 3 H), 2.97 (dd, 1 H), 3.26-3.51 (m, 3 H), 4.53 | | |
| | (m, 2 H), 6.75 (dd, 1 H), 7.05 (d, 1 H), 7.28 (d, 1 H), 7.54 (dd, 1 H), 7.77 (d, 1 H), 8.35 (dd, | | |
| | 1 H). | | |
| 14.20 | 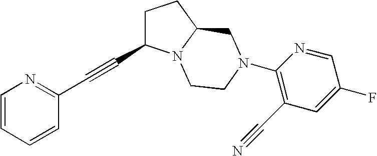 | (±)-5-fluoro-2-[(6R,8aS)-6-(pyridin-2-ylethynyl)hexahydropyrrolo[1,2-a]pyrazin-2(1H)-yl]nicotinonitrile | 65% |
| NMR | 1.63 (m, 1 H), 1.92-2.38 (m, 5 H), 2.94 (dd, 1 H), 3.21-3.50 (m, 3 H), 4.24 (m, 2 H), 7.36- | | |
| | 7.71 (m, 5 H), 8.25 (d, 1 H). | | |
| 14.21 | 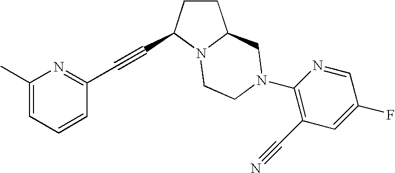 | (±)-5-fluoro-2-[(6R,8aS)-6-[(6-methylpyridin-2-yl)ethynyl]hexahydropyrrolo[1,2-a]pyrazin-2(1H)-yl]nicotinonitrile | 40% |
| NMR | 1.63 (m,1 H), 1.92-2.38 (m, 5 H), 2.58 (s, 3 H), 2.97 (dd, 1 H), 3.21-3.56 (m, 3 H), 4.3 (m, | | |
| | 2 H), 7.1 (d, 1), 7.53-7.65 (m, 3 H), 8.26 (d, 1 H). | | |
| 14.22 | 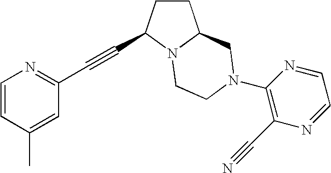 | (±)-3-[(6R,8aS)-6-[(4-methylpyridin-2-yl)ethynyl]hexahydropyrrolo[1,2-a]pyrazin-2(1H)-yl]pyrazine-2-carbonitrile | 38% |
| NMR | 1.65 (m, 1 H), 1.95-2.38 (m, 5 H), 2.36 (s, 3 H), 3.03 (dd, 1 H), 3.28-3.55 (m, 3 H), 4.57 | | |
| | (m, 2 H), 7.08 (d, 1 H), 7.31 (s, 1 H), 8.01 (d, 1 H), 8.25 (d, 1 H), 8.50 (d, 1 H). | | |
| 14.23 | 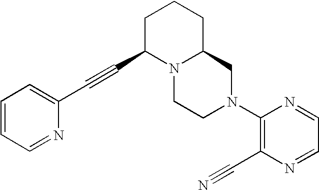 | (±)-3-[(6R,8aS)-6-(pyridin-2-yl-ethynyl)octahydro-2H-pyrido[1,2-a]pyrazin-2-yl]pyrazine-2-carbonitrile | 54% |
| NMR | 1.42 (m, 2 H), 1.73-2.34 (m, 6 H), 2.98 (dd, 1 H), 3.1 (dd, 1 H), 3.35 (dt, 1 H), 3.78 (m, | | |
| | 1 H), 4.35-4.56 (m, 2 H), 7.24 (dd, 1 H), 7.45 (d, 1 H), 7.63 (dd, 1 H), 8.01 (d, 1 H), 8.25 (d, | | |
| | 1 H), 8.58 (dd, 1 H). | | |
| 14.24 | 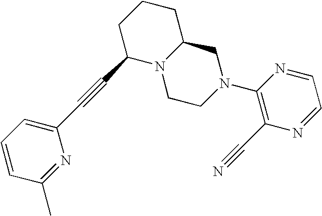 | (±)-3-{(6R,9aS)-6-[(6-methylpyridin-2-yl)ethynyl]octahydro-2H-pyrido[1,2-a]pyrazin-2-yl}pyrazine-2-carbonitrile | 54% |
| NMR | 1.40 (m, 2 H), 1.66-2.31 (m, 6 H), 2.56 (s, 3 H), 2.97 (dd, 1 H), 3.1 (dd, 1 H), 3.35 (dt, 1 H), | | |
| | 3.76 (m, 1 H), 4.35-4.54 (m, 2 H), 7.09 (d, 1 H), 7.26 (d, 1 H), 7.54 (dd, 1 H), 8.00 (d, 1 H), | | |
| | 8.25 (d, 1 H). | | |
| 14.25 | 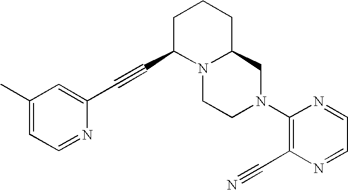 | (±)-3-{(6R,9aS)-6-[(4-methylpyridin-2-yl)ethynyl]octahydro-2H-pyrido[1,2-a]pyrazin-2-yl}pyrazine-2-carbonitrile | 59% |
| NMR | 1.43 (m, 2 H), 1.7-2.30 (m, 6 H), 2.35 (s, 3 H), 2.96 (dd, 1 H), 3.1 (dd, 1 H), 3.35 (dt, 1 H), | | |
| | 3.77 (m, 1 H), 4.35-4.56 (m, 2 H), 7.05 (d, 1 H), 7.28 (s, 1 H), 8.00 (d, 1 H), 8.25 (d, 1 H), | | |
| | 8.42 (d, 1 H). | | |
| 14.26 | 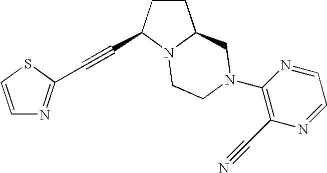 | (±)-3-[(6R,8aS)-6-(1,3-thiazol-2-yl-ethynyl)hexahydropyrrolo[1,2-a]pyrazin-2(1H)-yl]pyrazine-2-carbonitrile | 21% |
| NMR | 1.67 (m, 1 H), 1.91-2.37 (m, 5 H), 3.02 (dd, 1 H), 3.29-3.52 (m, 3 H), 4.60 (m, 2 H), | | |
| | 7.36 (d, 1 H), 7.82 (d, 1 H), 8.03 (d, 1 H), 8.27 (d, 1 H). | | |
| 14.27 | 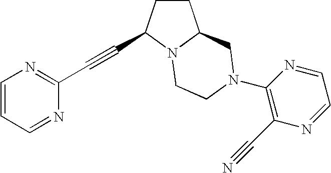 | (±)-3-[(6R,8aS)-6-(pyrimidin-2-yl-ethynyl)hexahydropyrrolo[1,2-a]pyrazin-2(1H)-yl]pyrzine-2-carbonitrile | 72% |
| NMR | 1.69 (m, 1 H), 1.93-2.37 (m, 5 H), 2.99 (dd, 1 H), 3.28-3.56 (m, 3 H), 4.58 (m, 2 H), | | |
| | 7.26 (d, 1 H), 8.02 (d, 1 H), 8.26 (d, 1 H), 8.72 (d, 1 H). | | |
| 14.28 | 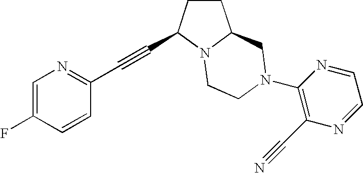 | (±)-3-[(6R,8aS)-6-[(5-fluoropyridin-2-yl)ethynyl]hexahydropyrrolo[1,2-a]pyrazin-2(1H)-yl]pyrazine-2-carbonitrile | 60% |
| NMR | 1.69 (m, 1 H), 1.93-2.4 (m, 5 H), 3.01 (dd, 1 H), 3.26-3.52 (m, 3 H), 4.61 (m, 2 H), 7.38 | | |
| | (d, 1 H), 7.81 (dd, 1 H), 8.02 (d, 1 H), 8.26 (d, 1 H), 8.64 (d, 1 H). | | |
| 14.29 | 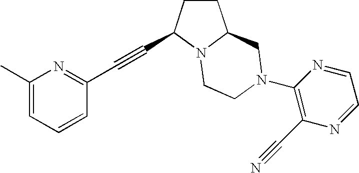 | (±)-3-[(6R,8aS)-6-[(6-methylpyridin-2-yl)ethynyl]hexahydropyrrolo[1,2-a]pyrazin-2(1H)-yl]pyrazine-2-carbonitrile | 62% |
| NMR | 1.67 (m, 1 H), 1.90-2.38 (m, 5 H), 2.56 (s, 3 H), 3.01 (dd, 1 H), 3.26-3.54 (m, 3 H), 4.59 | | |
| | (m, 2 H), 7.1 (d, 1 H), 7.3 (dd, 1 H), 7.57 (dd, 1 H), 8.01 (d, 1 H), 8.26 (d, 1 H). | | |
| 14.30 | 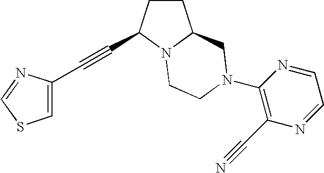 | (±)-3-[(6R,8aS)-6-(1,3-thiazol-4-ylethynyl)hexahydyropyrrolo[1,2-a]pyrazin-2(1H)-pyrazine-2-carbonitrile | 29% |

| | |
|---|---|
| NMR | 1.68 (m, 1 H), 1.91-2.50 (m, 5 H), 3.02 (dd, 1 H), 3.25-3.54 (m, 3 H), 4.60 (m, 2 H), |
| | 7.52 (d, 1 H), 8.02 (d, 1 H), 8.26 (d, 1 H), 8.78 (d, 1 H). |

### Example 15

### (±)-2-[(6R,8aS)-6-[(3-chlorophenyl)ethynyl]hexahydropyrrolo[1,2-a]pyrazin-2(1H)-yl]nicotinic acid

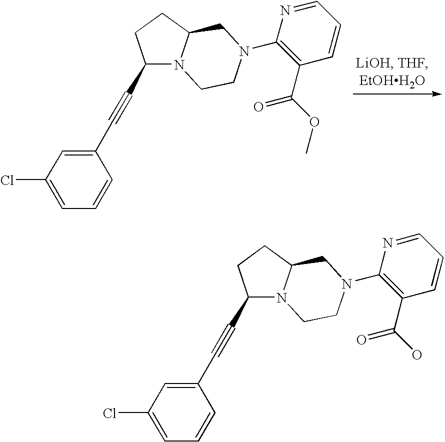

A mixture of (±)-methyl 2-[(6R,8aS)-6-[(3-chlorophenyl)ethynyl]hexahydropyrrolo[1,2-a]pyrazin-2(1H)-yl]nicotinate (160 mg, 0.4 mmol), LiOH (48 mg, 2 mmol), THF (1 mL), EtOH (1 mL) and H₂O (1 mL) was stirred overnight at RT and at 40° C. for 3 h. To this resulting mixture was added HCl aq (1N, 2 mL). After removal of solvent in vacuo, the residue was dissolved with DCM and filtered. The filtrate was concentrated and dried with vacuum pump to afford the product (148 mg, 94%). ¹H NMR 300 MHz, (CD₃OD): δ (ppm) 1.65-2.75 (m, 6H), 3.06 (dd, 1H), 3.33-4.15 (m, 5H), 6.98 (dd, 1H), 7.38 (m, 3H), 7.47 (s, 1H), 8.12 (d, 1H), 8.31 (d, 1H).

### Example 16

### (±)-2-[(6R,8aS)-6-[(3-chlorophenyl)ethynyl]hexahydropyrrolo[1,2-a]pyrazin-2(1H)-yl]nicotinamide

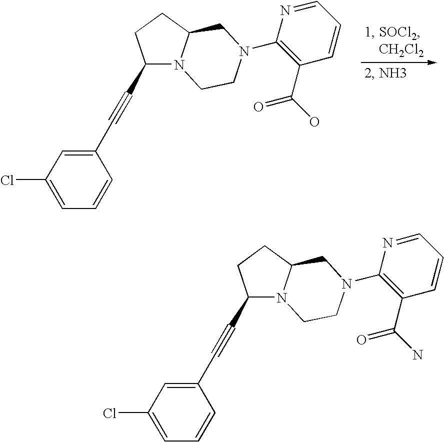

A mixture of (±)-2-[(6R,8aS)-6-[(3-chlorophenyl)ethynyl]hexahydropyrrolo[1,2-a]pyrazin-2(1H)-yl]nicotinic acid (30 mg, 0.08 mmol), SOCl₂ (70 mg, 0.6 mmol) and DCM (1 mL) was stirred at RT overnight and at 50° C. for 2 h. The resulting mixture was concentrated and was diluted with DCM (1 mL) and was cooled to −50° C. To this was added Et₃N (0.2 mL) and NH₃ (0.5N in dioxane, 1.5 mL). After stirring at RT for 1 h, the resulting mixture was washed with Na₂CO₃ aq (sat.), dried, concentrated and purified on silica gel column to afford product (18 mg, 60%). ¹H NMR 300 MHz, (CDCl₃) δ (ppm) 1.64-2.38 (m, 6H), 2.99 (dd, 1H), 3.24-3.65 (m, 5H), 6.07 (broad, 1H), 7.08 (dd, 1H), 7.28 (m, 3H), 7.45 (s, 1H), 8.28 (d, 1H), 8.3 (broad, 1H), 8.41 (d, 1H).

### Example 17

### (±)-(6R,8aS)-6-[(3-chlorophenyl)ethynyl]-2-[3-(2H-tetrazol-5-yl)pyridin-2-yl]octahydropyrrolo[1,2-a]pyrazine

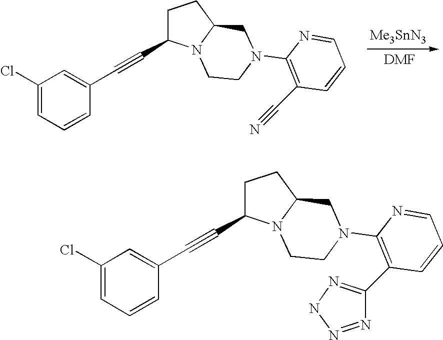

A mixture of (±)-2-[(6R,8aS)-6-[(3-chlorophenyl)ethynyl]hexahydropyrrolo[1,2-a]pyrazin-2(1H)-yl]nicotinonitrile (70 mg, 0.2 mmol), Me₃SnN₃ (160 mg, 0.8 mmol) and DMF was stirred at 90° C. overnight. To this resulting mixture was added water and extracted with ethyl acetate. After the solvent was removed in vacuo, the crude product was purified on silica gel column to afford the product (44 mg, 55%). ¹H NMR 300 MHz, (CDCl₃) δ (ppm): 1.65-2.5 (m, 6H), 3.04 (dd, 1H), 3.25-3.46 (m, 5H), 7.1 (dd, 1H), 7.24 (m, 3H), 7.4 (s, 1H), 8.29 (d, 1H), 8.41 (d, 1H).

### Example 18.1

### (±)-3-{(6R,9aS)-6-[(3-chlorophenyl)ethynyl]octahydro-2H-pyrido[1,2-a]pyrazin-2-yl}pyrazine-2-carbonitrile

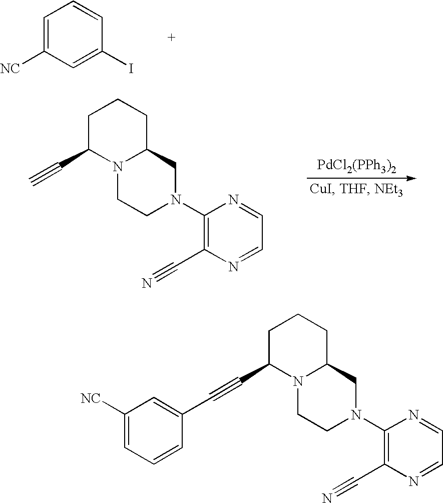

Bis(triphenylphosphine)palladium(II) dichloride (0.018 mmol) and copper(I) iodide (0.03 mmol) were added to a microwave vial with a stir bar. 3-iodobenzonitrile (0.45 mmol) and (±)-3-[(6R,9aS)-6-ethynyloctahydro-2H-pyrido[1,2-a]pyrazin-2-yl]pyrazine-2-carbonitrile (0.3 mmol, 80.2 mg) was dissolved in THF (1 mL) and added to the microwave vial with stirring, followed by Et₃N (1 mL). The vial was sealed and microwaved at 90 C for 6 min. The resulting mixture was then diluted with DCM and washed with water. The organic phase was purified by column chromatography to yield the desired product (80.3 mg, 73%). ¹H NMR 300 MHz, (CDCl₃) δ (ppm): 1.41 (m, 2H); 1.72 (m, 1H); 1.87 (m, 2H); 2.12 (m, 1H); 2.21 (m, 1H); 2.31 (td, 1H); 2.95 (dd, 1H); 3.09 (dd, 1H); 3.33 (td, 1H); 3.69 (d, 1H); 4.35 (d, 1H); 4.51 (d, 1H); 7.43 (t, 1H); 7.58 (d, 1H); 7.64 (d, 1H); 7.69 (s, 1H); 8.00 (d, 1H); 8.25 (d, 1H).

In a similar manner the following compounds were synthesized (bromo-pyridines were used in place of iodobenzenes for alkynyl pyridyl compounds):

| Example | Structure | Name | Yield |
|---|---|---|---|
| 18.2 | 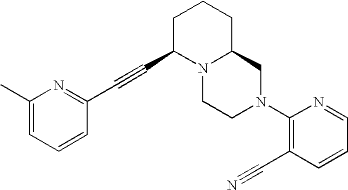 | (±)-2-{(6R,9aS)-6-[(6-methylpyridin-2-yl)ethynyl]octahydro-2H-pyrido[1,2-a]pyrazin-2-yl}nicotinonitrile | 50% |
| NMR | 1.42 (m, 2 H); 1.71 (m, 1 H); 1.87 (m, 2 H); 2.12 (m, 2 H); 2.35 (td, 1 H); 2.56 (s, 3 H); | | |
| | 2.92 (dd, 1 H); 3.14 (dd, 1 H); 3.30 (td, 1 H); 3.74 (d, 1 H); 4.26 (d, 1 H); 4.41 (d, 1 H); | | |
| | 6.74 (dd, 1 H); 7.09 (dd, 1 H); 7.27 (d, 1 H); 7.53 (t, 1 H); 7.75 (dd, 1 H); 8.34 (dd, 1 H). | | |
| 18.3 | 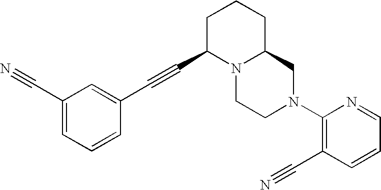 | (±)-2-{(6R,9aS)-6-[(3-cyanophenyl)ethynyl]octahydro-2H-pyrido[1,2-a]pyrazin-2-yl}nicotinonitrile | 38% |
| NMR | 1.43 (m, 2 H); 1.72 (m, 1 H); 1.87 (m, 2 H); 2.12 (m, 1 H); 2.21 (m, 1 H); 2.36 (td, 1 H); | | |
| | 2.93 (dd, 1 H); 3.11 (dd, 1 H); 3.30 (td, 1 H); 3.69 (d, 1 H); 4.25 (d, 1 H); 4.42 (d, 1 H); | | |
| | 6.77 (dd, 1 H); 7.43 (t, 1 H); 7.60 (d, 1 H); 7.66 (d, 1 H); 7.72 (s, 1 H); 7.78 (dd, 1 H); 8.36 | | |
| | (dd, 1 H). | | |
| 18.4 | 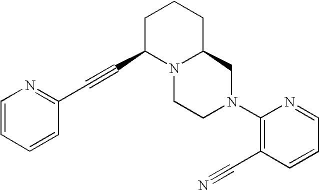 | (±)-2-{(6R,9aS)-6-(pyridin-2-yl-ethynyl)octahydro-2H-pyrido[1,2-a]pyrazin-2-yl}nicotinonitrile | 7% |
| NMR | 1.44 (m, 2 H); 1.66 (m, 1 H); 1.88 (m, 2 H); 2.12 (m, 2 H); 2.37 (td, 1 H); 2.94 (dd, 1 H); | | |
| | 3.16 (dd, 1 H); 3.32 (td, 1 H); 3.76 (d, 1 H); 4.14 (d, 1 H); 4.26 (d, 1 H); 6.76 (dd, 1 H); | | |
| | 7.25 (dd, 1 H); 7.45 (d, 1 H); 7.65 (td, 1 H); 7.78 (dd, 1 H); 8.36 (dd, 1 H); 8.58 (m, 1 H). | | |
| 18.5 | 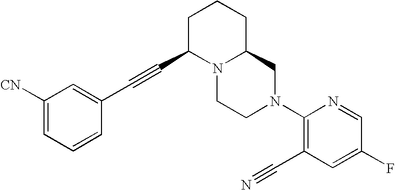 | (±)-2-{(6R,9aS)-6-[(3-cyanophenyl)ethynyl]octahydro-2H-pyrido[1,2-a]pyrazin-2-yl}-5-fluoronicotinonitrile | 66% |
| NMR | 1.40 (m, 2 H); 1.65 (m, 1 H); 1.84 (m, 2 H); 2.12 (m, 1 H); 2.23 (m, 1 H); 2.34 (td, 1 H); | | |
| | 2.88 (dd, 1 H); 3.07 (dd, 1 H); 3.25 (td, 1 H); 3.66 (d, 1 H); 4.02 (d, 1 H); 4.19 (d, 1 H); | | |
| | 7.41 (t, 1 H); 7.54 (m, 2 H); 7.63 (d, 1 H); 7.68 (s, 1 H); 8.22 (m, 1 H). | | |
| 18.6 | 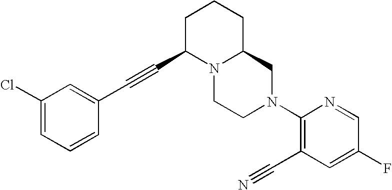 | (±)-2-{(6R,9aS)-6-[(3-chlorophenyl)ethynyl]octahydro-2H-pyrido[1,2-a]pyrazin-2-yl}-5-fluoronicotinonitrile | 60% |
| NMR | 1.41 (m, 2 H); 1.68 (m, 1 H); 1.85 (m, 2 H); 2.10 (m, 1 H); 2.24 (m, 1 H); 2.35 (td, 1 H); | | |
| | 2.88 (dd, 1 H); 3.08 (dd, 1 H); 3.27 (td, 1 H); 3.70 (d, 1 H); 4.05 (d, 1 H); 4.22 (d, 1 H); | | |
| | 7.24 (m, 3 H); 7.42 (s, 1 H); 7.52 (dd, 1 H); 8.25 (m, 1 H). | | |
| 18.7 | 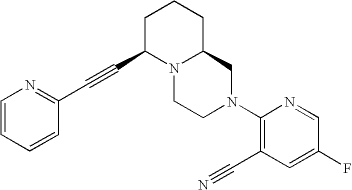 | (±)-5-fluoro-2-[(6R,9aS)-6-(pyridin-2-ylethynyl)octahydro-2H-pyrido[1,2-a]pyrazin-2-yl]nicotinonitrile | 35% |
| NMR | 1.41 (m, 2 H); 1.65 (m, 1 H); 1.85 (m, 2 H); 2.15 (m, 1 H); 2.24 (m, 1 H); 2.35 (td, 1 H); | | |
| | 2.89 (dd, 1 H); 3.13 (dd, 1 H); 3.26 (td, 1 H); 3.74 (d, 1 H); 4.04 (d, 1 H); 4.19 (d, 1 H); | | |
| | 7.22 (m, 1 H); 7.42 (d, 1 H); 7.52 (dd, 1 H); 7.63 (td, 1 H); 8.23 (d, 1 H); 8.56 (m, 1 H). | | |
| 18.8 | 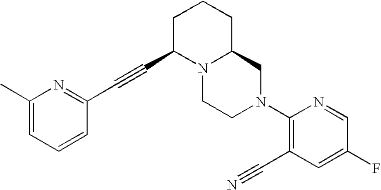 | (±)-5-fluoro-2-{(6R,9aS)-6-[(6-methylpyridin-2-yl)ethynyl]octahydro-2H-pyrido[1,2-a]pyrazin-2-yl}nicotinonitrile | 40% |

| | |
|---|---|
| NMR | 1.39 (m, 2 H); 1.64 (m, 1 H); 1.85 (m, 2 H); 2.13 (m, 1 H); 2.22 (m, 1 H); 2.33 (td, 1 H); |
| | 2.53 (s, 3 H); 2.88 (dd, 1 H); 3.11 (dd, 1 H); 3.25 (td, 1 H); 3.72 (d, 1 H); 4.04 (d, 1 H); 4.14 |
| | (d, 1 H); 7.07 (d, 1 H); 7.25 (t, 1 H); 7.51 (m, 2 H); 8.22 (d, 1 H). |

### Example 19

### 2-bromo-6-(fluoromethyl)pyridine

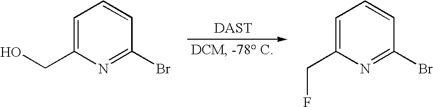

A cold solution of (6-bromopyridin-2-yl)methanol (3 g, 16 mmol) in DCM (50 mL) was added dropwise to a solution of DAST (7.85 g, 48 mmol) in DCM (70 mL) at −78° C. under stirring and nitrogen atmosphere. The resulting solution was stirred for additional 1 hr, then warmed to RT overnight. The reaction mixture was poured onto 300 ml ice cold water under stirring. The mixture was extracted with DCM (×3). The combined organic phase was washed with water and brine solution, dried over anhydrous sodium sulphate, concentrated under vacuum. The residue was purified by flash chromatography on silica gel eluting with 5-10% ethyl acetate in hexanes to give the title compound (2.4 g, 79%). ¹H NMR (400 MHz, CDCl₃): δ (ppm) 7.62 (1H, t), 7.45 (2H, m), 5.51 (1H, s), 5.40 (1H, s).

### Example 20.1

### (±)-3-[(6R,8aS)-6-[(6-methoxypyridin-2-yl)ethynyl]hexahydropyrrolo[1,2-a]pyrazin-2(1H)-yl]pyrazine-2-carbonitrile

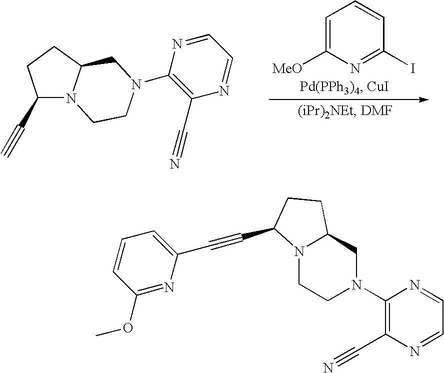

A mixture of 3-[(6R,8aS)-6-ethynylhexahydropyrrolo[1,2-a]pyrazin-2(1H)-yl]pyrazine-2-carbonitrile (300 mg, 1.18 mmol), 2-iodo-6-methoxypyridine (278 mg, 1.18 mmol), tetrakis(triphenylphosphine)palladium(0) (137 mg, 0.118 mmol), copper iodide (46 mg, 0.24 mmol), diisopropylethylamine (0.45 mL, 2.6 mmol) and DMF (40 mL) was stirred at RT overnight. 5% EDTA.Na₂.2H₂O (aq) (2 mL) was added and the reaction mixture was stirred at room for additional 30 min and then concentrated. Flash column chromatography gave the title compound (280 mg, 65%). ¹H NMR (400 MHz, CDCl₃): δ (ppm) 8.26 (d, 1H), 8.02 (d, 1H), 7.50 (t, 1H), 7.08 (d, 1H), 6.70 (d, 1H), 4.60 (t, 2H), 3.96 (s, 1H), 3.52 (d, 1H), 3.38-3.26 (m, 2H), 3.02 (t, 1H), 2.38-2.18 (m, 3H), 2.14-2.04 (m, 1H), 2.00-1.90 (m, 1H), 1.72-1.60 (m, 1H).

In a similar manner the following compounds were synthesized:

| Example | Structure | Name | Yield |
|---|---|---|---|
| 20.2 | 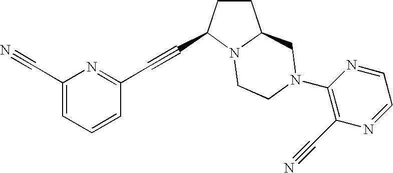 | (±)-3-[(6R,8aS)-6-[(6-cyanopyridin-2-yl)ethynyl]hexahydropyrrolo[1,2-a]pyrazin-2(1H)-yl]pyrazine-2-carbonitrile | 71% |
| NMR | 8.26 (d, 1H), 8.02 (d, 1H), 7.80 (t, 1H), 7.64 (dd, 2H), 4.60 (t, 2H), 3.48 (d, 1H), 3.36-3.26 (m, 2H), 3.04-2.96 | | |
| | (m, 1H), 2.40-2.20 (m, 3H), 2.14-2.06 (m, 1H), 2.00-1.92 (m, 1H), 1.70-1.60 (m, 1H). | | |
| 20.3 | 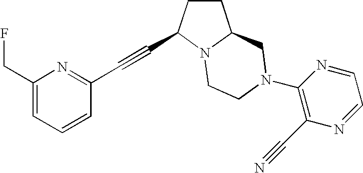 | (±)-3-[(6R,8aS)-6-{[6-(fluoromethyl)pyridin-2-yl]ethynyl}hexahydropyrrolo[1,2-a]pyrazin-2(1H)-yl]pyrazine-2-carbonitrile | 42% |

| | |
|---|---|
| NMR | 8.24 (d, 1H), 8.00 (d, 1H), 7.72 (t, 1H), 7.40 (d, 2H), 5.54 (s, 1H), 5.41 (s, 1H), 4.58 (m, 2H), 3.48 (d, 1H), |
| | 3.36-3.24 (m, 2H), 3.04-2.94 (m, 1H), 2.38-2.18 (m, 3H), 2.14-2.02 (m, 1H), 2.00-1.88 (m, 1H), 1.70-1.56 (m, 1H). |

### Example 21

### HPLC Separation of Enantiomers

| Example | Structure | Name |
|---|---|---|
| 21.1 | 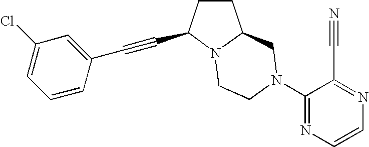 | 3-[(6R,8aS)-6-[(3-chlorophenyl)ethynyl]hexahydropyrrolo[1,2-a]pyrazin-2(1H)-yl]pyrazine-2-carbonitrile&3-[(6S,8aR)-6-[(3-chlorophenyl)ethynyl]hexahydropyrrolo[1,2-a]pyrazin-2(1H)-yl]pyrazine-2-carbonitrile |
| HPLC | Fraction 1: pale-yellow solid, 52 mg, Rt 10.6 min | |
| | Fraction 2: off-white solid, 56 mg, Rt 30.8 min | |
| | (Separation: Chiralpak AD 21 mm ID × 250 mm, 20 mic with MeOH at 20 mL/min; | |
| | analysis: 4.6 × 250 mm column; MeOH; 1 mL/min) | |
| 21.2 | 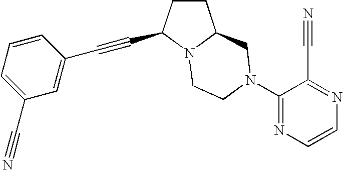 | 3-[(6R,8aS)-6-[(3-cyanophenyl)ethynyl]hexahydropyrrolo[1,2-a]pyrazin-2(1H)-yl]pyrazine-2-carbonitrile&3-[(6S,8aR)-6-[(3-cyanophenyl)ethynyl]hexahydropyrrolo[1,2-a]pyrazin-2(1H)-yl]pyrazine-2-carbonitrile |
| HPLC | Fraction 1: pale-yellow solid, 250 mg, Rt 11.8 min | |
| | Fraction 2: off-white solid, 250 mg, Rt 22.8 min | |
| | (Separation: Chiralpak AD 21 mm ID × 250 mm, 20 mic with MeOH at 20 mL/min; | |
| | analysis: 4.6 × 250 mm column, MeOH/EtOH = 5/95; 1 mL/min) | |
| 21.3 | 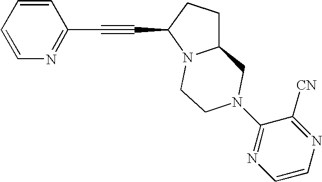 | 3-[(6R,8aS)-6-(pyridin-2-ylethynyl)hexahydro-pyrrolo[1,2-a]pyrazin-2(1H)-yl]pyrazine-2-carbonitrile&3-[(6S,8aR)-6-(pyridin-2-ylethynyl)hexahydro-pyrrolo[1,2-a]pyrazin-2(1H)-yl]pyrazine-2-carbonitrile |
| HPLC | Fraction 1: pale-yellow solid, 162 mg, Rt 8.4 min | |
| | Fraction 2: off-white solid, 245 mg, Rt 13.1 min | |
| | (Separation: Chiralpak AD 21 mm ID × 250 mm, 20 mic with MeOH at 20 mL/min; | |
| | analysis: 4.6 × 250 mm column, MeOH; 1 mL/min) | |
| 21.4 | 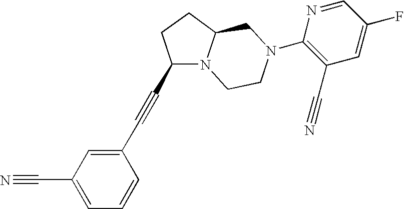 | 2-[(6R,8aS)-6-[3-cyanophenyl)ethynyl]hexahydropyrrolo[1,2-a]pyrazin-2(1H)-yl]-5-fluoronicotinonitrile&2-[(6S,8aR)-6-[(3-cyanophenyl)ethynyl]hexahydropyrrolo[1,2-a]pyrazin-2(1H)-yl]-5-fluoronicotinonitrile |
| HPLC | Fraction 1: beige solid, 78 mg, Rt 18.4 min | |
| | Fraction 2: beige solid, 68 mg, Rt 39.4 min | |
| | (Separation: Chiralpak AD 21 mm ID × 250 mm, 20 mic with 30% EtOH in MeOH | |
| | at 25 mL/min, the 100% EtOH at 20 mL/min after first peak eluted; analysis: 4.6 × | |
| | 250 mm column; 30% EtOH in MeOH; 1 mL/min) | |
| 21.5 | 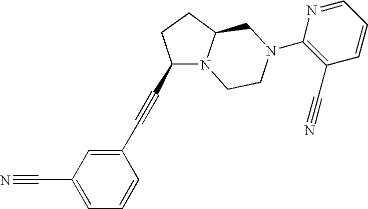 | 2-[(6R,8aS)-6-[(3-cyanophenyl)ethynyl]hexahydropyrrolo[1,2-a]pyrazin-2(1H)-yl]nicotinonitrile&2-[(6S,8aR)-6-[(3-cyanophenyl)ethynyl]hexahydropyrrolo[1,2-a]pyrazin-2(1H)-yl]nicotinonitrile |
| HPLC | Fraction 1: off-white solid, 176 mg, Rt 12.4 min | |
| | Fraction 2: off-white solid, 193 mg, Rt 23.4 min | |
| | (Separation: Chiralpak AD 21 mm ID × 250 mm, 20 mic with 30% EtOH in MeOH | |
| | at 25 mL/min; analysis: 4.6 × 250 mm column; 30% EtOH in MeOH; 1 mL/min) | |
| 21.6 | 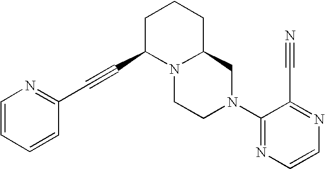 | 3-[(6R,9aS)-6-(pyridin-2-ylethynyl)octahydro-2H-pyrido[1,2-a]pyrazin-2-yl]pyrazine-2-carbonitrile&3-[(6S,9aR)-6-(pyridin-2-ylethynyl)octahydro-2H-pyrido[1,2-a]pyrazin-2-yl]pyrazine-2-carbonitrile |
| HPLC | Fraction 1: yellow sticky oil, 68 mg, Rt 8.4 min | |
| | Fraction 2: yellow sticky oil, 65 mg, Rt 18.6 min | |
| | (Separation: Chiralpak AD 21 mm ID × 250 mm, 20 mic with MeOH at 20 mL/min; | |
| | analysis: 4.6 × 250 mm column; MeOH; 1 mL/min) | |
| 21.7 | 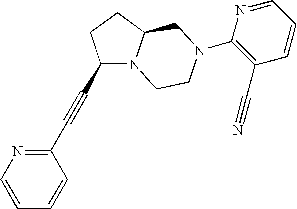 | 2-[(6R,8aS)-6-(pyridin-2-ylethynyl)hexahydropyrrolo[1,2-a]pyrazin-2(1H)-yl]nicotinonitrile&2-[(6S,8aR)-6-(pyridin-2-ylethynyl)hexahydropyrrolo[1,2-a]pyrazin-2(1H)-yl]nicotinonitrile |
| HPLC | Fraction 1: yellow sticky oil, 50 mg, Rt 7.3 min | |
| | Fraction 2: yellow sticky oil, 52 mg, Rt 23.4 min | |
| | (Separation: Chiralpak AD 21 mm ID × 250 mm, 20 mic with MeOH at 20 mL/min; | |
| | analysis: 4.6 × 250 mm column; MeOH; 1 mL/min) | |
| 21.8 | 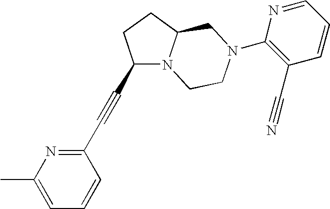 | 2-[(6R,8aS)-6-[(6-methylpyridin-2-yl)ethynyl]hexahydropyrrolo[1,2-a]pyrazin-2(1H)-yl]nictinonitrile&2-[(6S,8aR)-6-[(6-methylpyridin-2-yl)ethynyl]hexahydropyrrolo[1,2-a]pyrazin-2(1H)-yl]nictinonitrile |
| HPLC | Fraction 1: brown sticky oil, 86 mg, Rt 7.2 min | |
| | Fraction 2: brown sticky oil, 92 mg, Rt 10.0 min | |
| | (Separation: Chiralpak AD 21 mm ID × 250 mm, 20 mic with MeOH at 20 mL/min; | |
| | analysis: 4.6 × 250 mm column; MeOH; 1 mL/min) | |
| 21.9 | 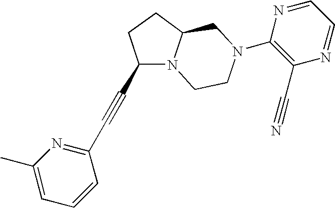 | 3-[(6R,8aS)-6-[(6-methylpyridin-2-yl)ethynyl]hexahydropyrrolo[1,2-a]pyrazin-2(1H)-yl]pyrazine-2-carbonitrile&3-[(6S,8aR)-6-[(6-methylpyridin-2-yl)ethynyl]hexahydropyrrolo[1,2-a]pyrazin-2(1H)-yl]pyrazine-2-carbonitrile |
| HPLC | Fraction 1: RT 16.08 min, 97.3%, yellowish solid | |
| | Fraction 2: RT 21.03 min, 99.9%, yellowish solid | |
| | Chiralpak_AD_20 mm × 250 mm, hexane/EtOH (9:1) | |
| 21.10 | 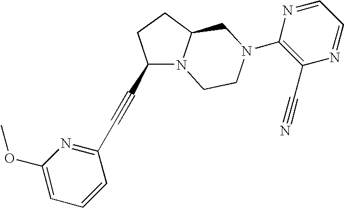 | 3-[(6R,8aS)-6-[(6-methoxypyridin-2-yl)ethynyl]hexahydropyrrolo[1,2-a]pyrazin-2(1H)-yl]pyrazine-2-carbonitrile&3-[(6S,8aR)-6-[(6-methoxypyridin-2-yl)ethynyl]hexahydropyrrolo[1,2-a]pyrazin-2(1H)-yl]pyrazine-2-carbonitrile |
| HPLC | Fraction 1: RT 17.59 min, 99.84%, yellowish solid | |
| | Fraction 2: RT 21.81 min, 99.83%, yellowish solid | |
| | Chiralpak_AD_20 mm × 250 mm, Hexane/EtOH 95/05 | |
| 21.11 | 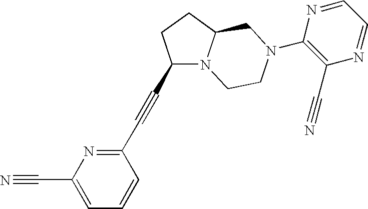 | 3-[(6R,8aS)-6-[(6-cyanopyridin-2-yl)ethynyl]hexahydropyrrolo[1,2-a]pyrazin-2(1H)-yl]pyrazine-2-carbonitrile&3-[(6S,8aR)-6-[(6-cyanopyridin-2-yl)ethynyl]hexahydropyrrolo[1,2-a]pyrazin-2(1H)-yl]pyrazine-2-carbonitrile |
| HPLC | Fraction 1: RT 24.27 min, 94.24%, (HPLC 90.24%) yellowish solid | |
| | Fraction 2: RT 49.42 min, 97.56%, yellowish solid | |
| | Chiralpak_AD_20 mm × 250 mm, Hexane/EtOH/iPrOH 75/15/10 | |
| 21.12 | 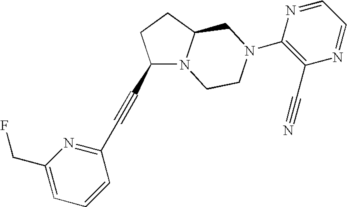 | 3-[(6R,8aS)-6-{[6-(fluoromethyl)pyridin-2-yl]ethynyl}hexahydropyrrolo[1,2-a]pyrazin-2(1H)-yl]pyrazine-2-carbonitrile&3-[(6S,8aR)-6-{[6-(fluoromethyl)pyridin-2-yl]ethynyl}hexahydropyrrolo[1,2-a]pyrazin-2(1H)-yl]pyrazine-2-carbonitrile |

| | |
|---|---|
| HPLC | Fraction 1: RT 16.06 min, 97.98%, yellowish solid |
| | Fraction 2: RT 27.50 min, 99.63%, yellowish solid |
| | Chiralpak_AD_20 mm × 250 mm, Hexane/EtOH 85/15 |

### Example 22

### Pharmaceutical Examples

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 min. in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol —CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP3). GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 mM) is added and incubated for an additional 30 min. The reaction is terminated by the addition of 0.5 ml perchloric acid (5%) on ice, with incubation at 4° C. for at least 30 min. Samples are collected in 15 ml polyproplylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

Generally, the compounds of the present invention were active in the assays described herein at concentrations (or with IC₅₀ values) of less than 10 μM. Preferred compounds of the invention have IC₅₀ values of less than 1 μM; more preferred compounds of less than about 100 nM. For example, the compounds of Examples 12.3, 13.3, 14.26, 13.12, 18.7 and 18.3 have IC₅₀ values of 187, 486, 439, 23, 83 and 20 DM, respectively.

What is claimed is:

1. A compound of formula I: 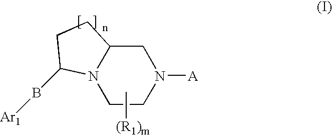
wherein:
Ar₁ is an optionally substituted aryl or heteroaryl group, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, —CN, CO₂R², SR², S(O)R², SO₂R², aryl, heteroaryl, cycloalkyl and heterocycloalkyl, wherein any cyclic group may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, —CN, CO₂R², SR², S(O)R² and SO₂R²;
A is selected from the group consisting of Ar₁, CO₂R², CONR²R³, S(O)R² and SO₂R²;
B is selected from the group consisting of vinylene and ethynylene, wherein the vinylene group is optionally substituted with up to 2 independently-selected C₁₋₆-alkyl groups;
R¹, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, (CO)R², O(CO)R², O(CO)OR², CO₂R², —CONR²R³, C₁₋₆-alkyleneOR², OC₂₋₆-allyleneOR² and C₁₋₆-allylenecyano;
R² and R³ are independently selected from the group consisting of H, C₁₋₆-alkyl, C₁₋₆-alkylhalo, C₂₋₆-alkenyl, C₂₋₆-alkynyl and cycloalkyl;
m is an integer selected from the group consisting of 0, 1, 2, 3 and 4; and
n is an integer selected from the group consisting of 1, 2 and 3;
or a pharmaceutically acceptable salt, hydrate, solvate, isoform, tautomer, optical isomer, or combination thereof

2. A compound according to claim 1 wherein B is an ethynylene group.

3. A compound according to claim 2 wherein Ar₁ is selected from the group consisting of an optionally-substituted phenyl group and an optionally-substituted pyridyl group.

4. A compound according to claim 3 wherein A is selected from the group consisting of an optionally-substituted pyridyl group and an optionally-substituted pyrazinyl group.

5. A compound according to claim 4 wherein A is selected from the group consisting of an optionally-substituted 2-pyridyl group and an optionally-substituted 2-pyrazinyl group

6. A compound selected from the group consisting of:
(±)-Methyl (6R,8aS)-6-[(3-chlorophenyl)ethynyl]hexahydropyrrolo[1,2-a]pyrazine-2(1H)-carboxylate,
(±)-ethyl (6R,9aS)-6-[(3-chlorophenyl)ethynyl]octahydro-2H-pyrido[1,2-a]pyrazine-2-carboxylate,
ethyl (6S,8aS)-6-[(3-chloro-phenyl)ethynyl]hexahydropyrrolo[1,2-a]pyrazine-2(1H)-carboxylate,
methyl (6S,8aS)-6-[(3-chloro-phenyl)ethynyl]hexahydropyrrolo[1,2-a]pyrazine-2(1H)-carboxylate,
(6S,8aS)—N-(2-chloroethyl)-6-[(3-chlorophenyl)ethynyl]hexahydropyrrolo[1,2-a]pyrazine-2(1H)-carboxamide,
(±)-ethyl (6R,8aS)-6-[(3-chloro-phenyl)ethynyl]hexahydropyrrolo[1,2-a]pyrazine-2(1H)-carboxylate,
(±)-ethyl (6R,9aS)-6-[(E)-2-(3-chlorophenyl)vinyl]octahydro-2H-pyrido[1,2-a]pyrazine-2-carboxylate,
(±)-3-[(6R,8aS)-6-[(3-chlorophenyl)ethynyl]hexahydropyrrolo[1,2-a]pyrazin-2(1H)-yl]pyrazine-2-carbonitrile,
(±)-6-{(6R,9aS)-6-[(3-chlorophenyl)ethynyl]octahydro-2H-pyrido[1,2-a]pyrazin-2-yl}nicotinonitrile,
6-[(6S,8aS)-6-[(3-chlorophenyl)ethynyl]hexahydropyrrolo[1,2-a]pyrazin-2(1H)-yl]nicotinonitrile,
2-[(6S,8aS)-6-[(3-chlorophenyl)ethynyl]hexahydropyrrolo[1,2-a]pyrazin-2(1H)-yl]nicotinonitrile,
(6S,8aS)-6-[(3-chlorophenyl)ethynyl]-2-(5-nitropyridin-2-yl) octahydropyrrolo[1,2-a]pyrazine,
2-[(6S,8aS)-6-[(3-chlorophenyl)ethynyl]hexahydropyrrolo[1,2-a]pyrazin-2(1H)-yl] isonicotinonitrile,
(±)-2-[(6R,8aS)-6-[(3-chlorophenyl)ethynyl]hexahydropyrrolo[1,2-a]pyrazin-2(1H)-yl]nicotinonitrile,
(±)-2-{(6R,9aS)-6-[(E)-2-(3-chloro-phenyl)vinyl]octahydro-2H-pyrido[1,2-a]pyrazin-2-yl}nicotinonitrile,
(±)-2-{(6R,9aS)-6-[(3-chlorophenyl)ethynyl]octahydro-2H-pyrido[1,2-a]pyrazin-2-yl}nicotinonitrile,
(±) -3-{(6R,9aS)-6-[(3-chlorophenyl)ethynyl]octahydro-2H-pyrido[1,2-a]pyrazin-2-yl}pyrazine-2-carbonitrile,
(±)-2-[(6R,8aS)-6-[(3-cyanophenyl)ethynyl]hexahydropyrrolo[1,2-a]pyrazin-2(1H)-yl]nicotinonitrile,
(±)-methyl 2-[(6R,8aS)-6-[(3-chloro-phenyl)ethynyl]hexahydropyrrolo[1,2-a]pyrazin-2(1H)-yl]nicotinate,
(±)-2-[(6R,8aS)-6-[(3-chlorophenyl)ethynyl]hexahydropyrrolo[1,2-a]pyrazin-2(1H)-yl]-5-fluoro-nicotinonitrile,
(±)-2-[(6R,8aS)-6-[(3-cyanophenyl)ethynyl]hexahydropyrrolo[1,2-a]pyrazin-2(1H)-yl]-5-fluoro-nicotinonitrile,
(±)-3-[(6R,8aS)-6-[(3-cyanophenyl)ethynyl]hexahydropyrrolo[1,2-a]pyrazin-2(1H)-yl]pyrazine-2-carbonitrile,
(±)-tert-butyl (6R,9aS)-6-[(3-chlorophenyl)ethynyl]octahydro-2H-pyrido[1,2-a]pyrazine-2-carboxylate,
(±)-3-[(6R,8aS)-6-[(2-chlorophenyl)ethynyl]hexahydropyrrolo[1,2-a]pyrazin-2(1H)-yl]pyrazine-2-carbonitrile,
(±)-3-[(6R,8aS)-6-[(3-methoxy-phenyl)ethynyl]hexahydropyrrolo[1,2-a]pyrazin-2(1H)-yl]pyrazine-2-carbonitrile,
(±)-3-[(6R,8aS)-6-[(2,4-dichloro-phenyl)ethynyl]hexahydropyrrolo[1,2-a]pyrazin-2(1H)-yl]pyrazine-2-carbonitrile,
(±)-3-[(6R,8aS)-6-[(5-chloro-2-fluorophenyl)ethynyl]hexahydropyrrolo[1,2-a]pyrazin-2(1H)-yl]pyrazine-2-carbonitrile,
(±)-3-[(6R,8aS)-6-(phenylethynyl) hexahydropyrrolo[1,2-a]pyrazin-2(1H)-yl]pyrazine-2-carbonitrile,
(±)-3-[(6R,8aS)-6-[(3-fluorophenyl)ethynyl]hexahydropyrrolo[1,2-a]pyrazin-2(1H)-yl]pyrazine-2-carbonitrile,
(±)-3-[(6R,8aS)-6-[(4-chlorophenyl)ethynyl]hexahydropyrrolo[1,2-a]pyrazin-2(1H)-yl]pyrazine-2-carbonitrile,
(±)-3-[(6R,8aS)-6-[(2-bromophenyl)ethynyl]hexahydropyrrolo[1,2-a]pyrazin-2(1H)-yl]pyrazine-2-carbonitrile,
(±)-3-[(6R,8aS)-6-[(3-bromophenyl)ethynyl]hexahydropyrrolo[1,2-a]pyrazin-2(1H)-yl]pyrazine-2-carbonitrile,
(±)-3-[(6R,8aS)-6-[(3,5-difluoro-phenyl)ethynyl]hexahydropyrrolo[1,2-a]pyrazin-2(1H)-yl]pyrazine-2-carbonitrile,
(±)-3-[(6R,8aS)-6-[(2,4-difluoro-phenyl)ethynyl]hexahydropyrrolo[1,2-a]pyrazin-2(1H)-yl]pyrazine-2-carbonitrile,
(±)-3-[(6R,8aS)-6-[(2,5-dichloro-phenyl)ethynyl]hexahydropyrrolo[1,2-a]pyrazin-2(1H)-yl]pyrazine-2-carbonitrile,
(±)-3-[(6R,8aS)-6-[(4-cyanophenyl)ethynyl]hexahydropyrrolo[1,2-a]pyrazin-2(1H)-yl]pyrazine-2-carbonitrile,
(±)-3-[(6R,8aS)-6-(pyridin-2-yl-ethynyl)hexahydropyrrolo[1,2-a]pyrazin-2(1H)-yl]pyrazine-2-carbonitrile,
(±)-3-[(6R,8aS)-6-[(5-cyanopyridin-3-yl)ethynyl]hexahydropyrrolo[1,2-a]pyrazin-2(1H)-yl]pyrazine-2-carbonitrile,
(±)-2-[(6R,8aS)-6-(pyridin-2-yl-ethynyl)hexahydropyrrolo[1,2-a]pyrazin-2(1H)-yl]nicotinonitrile,
(±)-2-[(6R,8aS)-6-[(6-methylpyridin-2-yl)ethynyl]hexahydropyrrolo[1,2-a]pyrazin-2(1H)-yl]nicotinonitrile,
(±)-5-fluoro-2-[(6R,8aS)-6-(pyridin-2-ylethynyl)hexahydropyrrolo[1,2-a]pyrazin-2(1H)-yl]nicotinonitrile,
(±)-5-fluoro-2-[(6R,8aS)-6-[(6-methylpyridin-2-yl)ethynyl]hexahydropyrrolo[1,2-a]pyrazin-2(1H)-yl]nicotinonitrile,
(±)-3-[(6R,8aS)-6-[(4-methylpyridin-2-yl)ethynyl]hexahydropyrrolo[1,2-a]pyrazin-2(1H)-yl]pyrazine-2-carbonitrile,
(±)-3-[(6R,9aS)-6-(pyridin-2-yl-ethynyl)octahydro-2H-pyrido[1,2-a]pyrazin-2-yl]pyrazine-2-carbonitrile,
(±)-3-{(6R,9aS)-6-[(6-methylpyridin-2-yl)ethynyl]octahydro-2H-pyrido[1,2-a]pyrazin-2-yl}pyrazine-2-carbonitrile,
(±)-3-{(6R,9aS)-6-[(4-methylpyridin-2-yl)ethynyl]octahydro-2H-pyrido[1,2-a]pyrazin-2-yl}pyrazine-2-carbonitrile,
(±)-3-[(6R,8aS)-6-(1,3-thiazol-2-yl-ethynyl)hexahydropyrrolo[1,2-a]pyrazin-2(1H)-yl]pyrazine-2-carbonitrile,
(±)-3-[(6R,8aS)-6-(pyrimidin-2-yl-ethynyl)hexahydropyrrolo[1,2-a]pyrazin-2(1H)-yl]pyrazine-2-carbonitrile,
(±)-3-[(6R,8aS)-6-[(5-fluoropyridin-2-yl)ethynyl]hexahydropyrrolo[1,2-a]pyrazin-2(1H)-yl]pyrazine-2-carbonitrile,
(±)-3-[(6R,18aS)-6-[(6-methylpyridin-2-yl)ethynyl]hexahydropyrrolo[1,2-a]pyrazin-2(1H)-yl]pyrazine-2-carbonitrile,
(±)-3-[(6R,8aS)-6-(1,3-thiazol-4-ylethynyl)hexahydropyrrolo[1,2-a]pyrazin-2(1H)-yl]pyrazine-2-carbonitrile,
(±)-2-[(6R,8aS)-6-[(3-chlorophenyl)ethynyl]hexahydropyrrolo[1,2-a]pyrazin-2(1H)-yl]nicotinic acid,
(±)-2-[(6R,8aS)-6-[(3-chlorophenyl)ethynyl]hexahydropyrrolo[1,2-a]pyrazin-2(1H)-yl]nicotinamide,
(±)-(6R,8aS)-6-[(3-chlorophenyl)ethynyl]-2-[3-(2H-tetrazol-5-yl)pyridin-2-yl]octahydropyrrolo[1,2-a]pyrazine,
(±)-3-{(6R,9aS)-6-[(3-chlorophenyl)ethynyl]octahydro-2H-pyrido[1,2-a]pyrazin-2-yl}pyrazine-2-carbonitrile,
(±)-2-{(6R,9aS)-6-[(6-methylpyridin-2-yl)ethynyl]octahydro-2H-pyrido[1,2-a]pyrazin-2-yl}nicotinonitrile,
(±)-2-{(6R,9aS)-6-[(3-cyanophenyl)ethynyl]octahydro-2H-pyrido[1,2-a]pyrazin-2-yl}nicotinonitrile,
(±)-2-{(6R,9aS)-6-(pyridin-2-yl-ethynyl)octahydro-2H-pyrido[1,2-a]pyrazin-2-yl}nicotinonitrile,
(±)-2-{(6R,9aS)-6-[(3-cyanophenyl)ethynyl]octahydro-2H-pyrido[1,2-a]pyrazin-2-yl}-5-fluoronicotinonitrile,
(±)-2-{(6R,9aS)-6-[(3-chlorophenyl)ethynyl]octahydro-2H-pyrido[1,2-a]pyrazin-2-yl}-5-fluoronicotinonitrile,
(±)-5-fluoro-2-[(6R,9aS)-6-(pyridin-2-ylethynyl)octahydro-2H-pyrido[1,2-a]pyrazin-2-yl]nicotinonitrile,
(±)-5-fluoro-2-{(6R,9aS)-6-[(6-methylpyridin-2-yl)ethynyl]octahydro-2H-pyrido[1,2-a]pyrazin-2-yl}nicotinonitrile,
(±)-3-[(6R,8aS)-6-[(6-methoxypyridin-2-yl)ethynyl]hexahydropyrrolo[1,2-a]pyrazin-2(1H)-yl]pyrazine-2-carbonitrile,
(±)-3-[(6R,8aS)-6-[(6-cyanopyridin-2-yl)ethynyl]hexahydropyrrolo[1,2-a]pyrazin-2(1H)-yl]pyrazine-2-carbonitrile,
(±)-3-[(6R,8aS)-6-{[6-(fluoromethyl)pyridin-2-yl]ethynyl}hexahydropyrrolo[1,2-a]pyrazin-2(1H)-yl]pyrazine-2-carbonitrile,
3-[(6R,8aS)-6-[(3-chlorophenyl)ethynyl]hexahydropyrrolo[1,2-a]pyrazin-2(1H)-yl]pyrazine-2-carbonitrile,
3-[(6S,8aR)-6-[(3-chlorophenyl)ethynyl]hexahydropyrrolo[1,2-a]pyrazin-2(1H)-yl]pyrazine-2-carbonitrile,
3-[(6R,8aS)-6-[(3-cyanophenyl)ethynyl]hexahydropyrrolo[1,2-a]pyrazin-2(1H)-yl]pyrazine-2-carbonitrile,
3-[(6S,8aR)-6-[(3-cyanophenyl)ethynyl]hexahydropyrrolo[1,2-a]pyrazin-2(1H)-yl]pyrazine-2-carbonitrile,
3-[(6R,8aS)-6-(pyridin-2-ylethynyl)hexahydro-pyrrolo[1,2-a]pyrazin-2(1H)-yl]pyrazine-2-carbonitrile,
3-[(6S,8aR)-6-(pyridin-2-ylethynyl)hexahydro-pyrrolo[1,2-a]pyrazin-2(1H)-yl]pyrazine-2-carbonitrile,
2-[(6R,8aS)-6-[(3-cyanophenyl)ethynyl]hexahydropyrrolo[1,2-a]pyrazin-2(1H)-yl]-5-fluoronicotinonitrile,
2-[(6S,8aR)-6-[(3-cyanophenyl)ethynyl]hexahydropyrrolo[1,2-a]pyrazin-2(1H)-yl]-5-fluoronicotinonitrile,
2-[(6R,8aS)-6-[(3-cyanophenyl)ethynyl]hexahydropyrrolo[1,2-a]pyrazin-2(1H)-yl]nicotinonitrile,
2-[(6S,8aR)-6-[(3-cyanophenyl)ethynyl]hexahydropyrrolo[1,2-a]pyrazin-2(1H)-yl]nicotinonitrile,
3-[(6R,9aS)-6-(pyridin-2-ylethynyl)octahydro-2H-pyrido[1,2-a]pyrazin-2-yl]pyrazine-2-carbonitrile,
3-[(6S,9aR)-6-(pyridin-2-ylethynyl)octahydro-2H-pyrido[1,2-a]pyrazin-2-yl]pyrazine-2-carbonitrile,
2-[(6R,8aS)-6-(pyridin-2-ylethynyl)hexahydropyrrolo[1,2-a]pyrazin-2(1H)-yl]nicotinonitrile,
2-[(6S,8aR)-6-(pyridin-2-ylethynyl)hexahydropyrrolo[1,2-a]pyrazin-2(1H)-yl]nicotinonitrile,
2-[(6R,8aS)-6-[(6-methylpyridin-2-yl)ethynyl]hexahydropyrrolo[1,2-a]pyrazin-2(1H)-yl]nicotinonitrile,
2-[(6S,8aR)-6-[(6-methylpyridin-2-yl)ethynyl]hexahydropyrrolo[1,2-a]pyrazin-2(1H)-yl]nicotinonitrile,
3-[(6R,8aS)-6-[(6-methylpyridin-2-yl)ethynyl]hexahydropyrrolo[1,2-a]pyrazin-2(1H)-yl]pyrazine-2-carbonitrile,
3-[(6S,8aR)-6-[(6-methylpyridin-2-yl)ethynyl]hexahydropyrrolo[1,2-a]pyrazin-2(1H)-yl]pyrazine-2-carbonitrile,
3-[(6R,8aS)-6-[(6-methoxypyridin-2-yl)ethynyl]hexahydropyrrolo[1,2-a]pyrazin-2(1H)-yl]pyrazine-2-carbonitrile,
3-[(6S,8aR)-6-[(6-methoxypyridin-2-yl)ethynyl]hexahydropyrrolo[1,2-a]pyrazin-2(1H)-yl]pyrazine-2-carbonitrile,
3-[(6R,8aS)-6-[(6-cyanopyridin-2-yl)ethynyl]hexahydropyrrolo[1,2-a]pyrazin-2(1H)-yl]pyrazine-2-carbonitrile,
3-[(6S,8aR)-6-[(6-cyanopyridin-2-yl)ethynyl]hexahydropyrrolo[1,2-a]pyrazin-2(1H)-yl]pyrazine-2-carbonitrile,
3-[(6R,8aS)-6-{[6-(fluoromethyl)pyridin-2-yl]ethynyl}hexahydropyrrolo[1,2-a]pyrazin-2(1H)-yl]pyrazine-2-carbonitrile, and
3-[(6S,8aR)-6-{[6-(fluoromethyl)pyridin-2-yl]ethynyl}hexahydropyrrolo[1,2-a]pyrazin-2(1H)-yl]pyrazine-2-carbonitrile.

7. A pharmaceutical composition comprising as active ingredient a therapeutically effective amount of the compound according to claim 1, in association with one or more pharmaceutically acceptable diluents, excipients and/or inert carriers.

8. The pharmaceutical composition according to claim 7, for use in the treatment of mGluR5-mediated disorders.

9. The compound according to claim 1 for use in therapy.

10. The compound according to claim 1 for use in treatment of mGluR5-mediated disorders.

11. Use of the compound according to claim 1, in the manufacture of a medicament for the treatment of mGluR5-mediated disorders.

12. A method of treatment of mGluR5-mediated disorders, comprising administering to a mammal a therapeutically effective amount of the compound according to claim 1.

13. The method according to claim 12, wherein the mammal is a human.

14. The method according to claim 12, wherein the disorders are neurological disorders.

15. The method according to claim 12, wherein the disorders are psychiatric disorders.

16. The method according to claim 12, wherein the disorders are chronic and acute pain disorders.

17. The method according to claim 12, wherein the disorders are gastrointestinal disorders.

18. A method for inhibiting activation of mGluR5 receptors, comprising treating a cell containing said receptor with an effective amount of the compound according to claim 1.
